(19) United States
(12) Patent Application Publication
(10) Pub. No.: US 2008/0015494 A1
(43) Pub. Date: Jan. 17, 2008

(54) MULTI-RESERVOIR PUMP DEVICE FOR DIALYSIS, BIOSENSING, OR DELIVERY OF SUBSTANCES

(75) Inventors: John T. Santini JR., North Chelmsford, MA (US); Michael J. Cima, Winchester, MA (US); Jonathan R. Coppeta, Windham, NH (US); James H. Prescott, Cambridge, MA (US); Zouhair Sbiaa, Everett, MA (US); Mark A. Staples, Cambridge, MA (US)

Correspondence Address: SUTHERLAND ASBILL & BRENNAN LLP, 999 PEACHTREE STREET, N.E., ATLANTA, GA, 30309, US

(73) Assignee: MICROCHIPS, INC., Bedford, MA (US)

(21) Appl. No.: 11/776,351

(22) Filed: Jul. 11, 2007

Related U.S. Application Data
(60) Provisional application No. 60/807,032, filed on Jul. 11, 2006.

(51) Int. Cl. A61M 31/00

(52) U.S. Cl. 604/65

(57) ABSTRACT

A pump patch device is provided for drug delivery. The device may include a substrate having a plurality of discrete reservoirs, each reservoir having a reservoir opening; a drug disposed in the reservoirs; a pump for delivering a carrier fluid through or adjacent to the reservoir openings; a flow channel for receiving and combining the carrier fluid from the pump with the drug from the reservoirs to form a fluidized drug; and a needle for delivering the fluidized drug into the skin or another biological tissue of a patient. A device is provided for use in dialysis that includes a non-disposable module including a pump or pressure generator; and a disposable cassette operably connected to the pump or pressure generator and including a plurality of discrete reservoirs containing drug and sensors. A fluidics connection device is provided that includes a compression cold weld seal for a microfluidic via.

### CROSS-REFERENCE TO RELATED APPLICATIONS

The present application claims benefit of U.S. Provisional Application No. 60/807,032, filed Jul. 11, 2006. That application is incorporated herein by reference.

### BACKGROUND OF THE INVENTION

This invention relates generally to miniaturized devices for controlled delivery of chemicals, for sensing, for purification processes, or for a combination thereof, and more particularly to medical devices for drug delivery, biosensing, and dialysis.

Accurate delivery of small, precise quantities of chemicals at a delivery site is of great importance in many different fields of science and industry. Examples in medicine include the delivery of drugs to patients, for example by intravenous, transdermal, or pulmonary administration methods. Examples in diagnostics include releasing reagents into fluids to conduct DNA or genetic analyses, combinatorial chemistry, or detection of specific molecules in environmental samples. These delivery systems often involve the use of a pump.

Pumps have been used in various ex vivo fluid delivery applications. For instance, pumps can be connected to a patient by an intravenous line/needle/catheter, by transdermal needles/microneedles, or by a permanent access port (e.g., for peritoneal dialysis). Pumps may be adapted for hospital, clinic, or home use, depending on the size, complexity, cost, and frequency of use of the unit. Generally, pumps can be used to deliver fluid drugs continuously (e.g., zero-order or basal delivery) or in a pulsatile manner.

Various conventional pumping mechanisms have been used, but each one has its limitations or disadvantages. For example, displacement pumps, such as syringe and peristaltic pumps, deliver a certain volume of fluid per unit time or per cycle, respectively. A syringe pump, where the volume is being delivered in a single stroke, however, is spatially inefficient because it wastes twice the volume of the drug solution to be delivered due to the plunger position when the syringe is filled. That is, the space necessarily occupied by the plunger in the reservoir cannot be used to hold drug. While a piston pump is more spatially efficient, it is at the cost of requiring multiple strokes and greater device complexity. Reciprocating piston pumps may require complex mechanical structures and many moving parts, or they may be too large and expensive to be incorporated into a disposable device. Conventional osmotic pumps cannot be actively controlled to selectively vary the flow rates on command. Electrophoretic pumps have flow rates which are highly dependent on the composition of the drug solution (i.e., concentration and ionic strength). MEMS pumps usually include membrane or diaphragm actuators, so pump operation can be significantly affected by the presence of air bubbles.

Conventional pumps generally deliver drugs in only in a liquid form. In many cases, however, it may be undesirable to rely on liquid drug forms, because of the short shelf life or instability of certain drugs in liquid form. For example, certain protein drugs are far more shelf stable at room temperature in solid form (e.g., lyophilized) rather than in solution form (e.g., in a physiologically acceptable liquid vehicle). In addition, certain drug solutions or suspensions may be incompatible with the materials of construction of the pump. For instance, the drug solution may be corrosive to pump materials, or the pump materials undesirably may cause drug to aggregate or precipitate from solution.

In one approach, a medical device may include a pumping mechanism that operates by using a pressurized reservoir to deliver a dose of drug by metering out a volume of a drug solution of known concentration. One type of pressurized reservoir pump is an elastic bladder. For example, U.S. Pat. No. 3,469,578 to Bierman, U.S. Pat. No. 4,318,400 to Perry, and U.S. Pat. No. 5,016,047 to Kriesel describe devices that incorporate elastic bladders, which contract to expel their drug contents. The volume of drug solution ejected from an orifice in the reservoir—and thus the delivered dose of drug—is dependent on several parameters including the pressure in the reservoir, the length of the flow tube, the inside diameter of the flow tube, and the viscosity of the fluid being delivered, which may be dependent on the temperature of the fluid. Therefore, the pressure in devices using an elastic bladder decreases over time. This can make it difficult to finely control drug dosing.

To control flow using pressurized reservoirs with conventional pumping devices, it has been necessary to include some combination of valves, sensors (e.g., to measure pressure, flow, viscosity, and/or temperature), complex algorithms, and/or other means to compensate for the pressure loss over time. For example, techniques for reducing the pressure variation in fluid flowing from such reservoir devices are described in U.S. Pat. No. 4,447,224 to Idriss (describing flow resistors), U.S. Pat. No. 4,741,736 and No. 4,447,232 to Sealfon and U.S. Pat. No. 5,248,300 to Bryant (describing constant force springs), in U.S. Pat. No. 5,061,242 to Sampson and U.S. Pat. No. 5,665,070 to McPhee (describing other devices for reducing the variability of reservoir pressurization in elastic bladder pumps to maintain constant drug infusion rates), and U.S. Pat. No. 6,582,393 to Sage (describing maintaining accurate dosing of liquid drugs by automatically changing the time that a flow valve is open in order to compensate for changing reservoir pressures). However, if it were desired to modulate drug dosing over time, such as delivering drugs only at predetermined intervals or in a pulsatile manner, the foregoing devices would require actively controllable valves or flow restricting technology, which would add additional complexity and cost to these devices.

In another type of pressurized reservoir pump, the reservoir is pressurized by the generation of gas, which serves to move a membrane or piston, as disclosed for example, in U.S. Pat. No. 6,939,324 to Gonnelli and U.S. Pat. No. 5,527,288 to Gross. The membrane or piston may be flexible or rigid. The volume of drug solution delivered to the patient is proportional to the amount of gas generated. The gas may be generated by an electrochemical cell, for example. However, because gases are compressible, the reservoir pressure resulting from a given mass of generated gas may vary during operation and would be affected by the temperature of the gas and the viscosity (and temperature) of the liquid to be delivered. The liquid may also have a non-Newtonian viscosity which further complicates the relationship between pressure and flow rates. The resulting flow also may depend on the physical dimensions of the pump, including the length and inside diameter of the flow tube.

Pumping mechanisms have been incorporated into a number of proposed or commercial medical devices. For example, U.S. Pat. No. 5,989,423 to Kamen describes a disposable cassette for peritoneal dialysis using flexible diaphragms as valves to direct fluid flow through the cassette and a pneumatic pumping mechanism. MiniMed (now part of Medtronic) developed externally worn insulin pumps, and Alza developed an implantable micro-osmotic pump for delivering solutions. Debiotech developed the NANOPUMP™, which is a miniaturized drug delivery, volumetric membrane pump device. Biovalve Inc. reportedly has developed a transdermal release, disposable micropump system. All of these devices, however, include one or more of the limitations and disadvantages associated with conventional pumping mechanisms as described above.

It therefore would be desirable to provide relatively simple pumping devices for delivering drug or other chemicals that overcome the shortcomings and limitations associated with conventional pressurized reservoir pump systems. It would also be desirable to provide drug delivery devices capable of storing drug in a solid form, and then delivering drug (e.g., in fluid form) continually or in a pulsatile manner. It would be particularly desirable for the device to deliver accurate dosages of drug without waste and preferably without numerous valves or other moving parts. Desirably, the device would be inexpensive enough to manufacture and use so that it could be at least in part disposable following delivery of drug, particularly where the drug can stored in its most stable form locally in the device.

### SUMMARY OF THE INVENTION

In one aspect, a pump patch device is provided for the delivery of a drug to a patient in need thereof. In one embodiment the device include a substrate which includes a plurality of discrete reservoirs, each reservoir having at least one reservoir opening; a drug disposed in the reservoirs; a pump for delivering a carrier fluid through or adjacent to the at least one opening of each of the reservoirs; a flow channel for receiving and combining the carrier fluid from the pump and the drug from at least one of the reservoirs to form a fluidized drug; and at least one needle for delivering the fluidized drug into the skin or another biological tissue of the patient. In one embodiment, the device includes a housing for the substrate, the drug the pump, the flow channel, the at least one needle, and a source of carrier fluid. The device may further include an adhesive material or other securement feature for releasably securing the device to the skin or other biological tissue surface.

In one embodiment, the device further includes a first plurality of discrete reservoir caps, each cap closing the at least one reservoir opening of each reservoir. The device of this embodiment may further include a controller and a power source for disintegrating the first plurality of reservoir caps to initiate mixing of the drug with the carrier fluid. The controller and the power source may be part of a reusable module which can be releasably secured to a drug reservoir array module, which includes the substrate, the drug the pump, the flow channel, the needle, and a source of carrier fluid. The needle may be in the form a one or more microneedles. In various embodiments, the pump may include a pressurized reservoir, a gas generation mechanism, a syringe pump, or a peristaltic pump. The drug in the reservoirs may be in a solid or gel formulation.

In one embodiment, each of the drug-containing reservoirs includes a second reservoir opening, and these second reservoir openings are closed by a second plurality of reservoir caps. In a certain embodiment, the device further includes a second flow channel wherein the carrier fluid from the pump can flow through a reservoir, once the reservoir caps closing the first and second reservoir openings of the reservoir have been disintegrated.

In an embodiment of the pump patch device, the pump may include a carrier fluid reservoir which can be pressurized to drive carrier fluid through the flow channel. The device may further include a separate pressure manifold with a flexible membrane which, following disintegration of the reservoir cap closing the at least one reservoir opening, pushes against the drug from the side of the reservoir opposed to the reservoir opening in order to displace the drug from the reservoir.

In another aspect, a method is provided for delivering a drug into the skin or another biological tissue of a patient. In one embodiment, the method includes: (a) providing a pump patch device that comprises (i) a substrate which includes a plurality of discrete reservoirs, each reservoir having at least one reservoir opening; (ii) a drug disposed in the reservoirs; (iii) a pump comprising a carrier fluid supply, (iv) a flow channel, and (v) at least one needle; (b) inserting the needle into the patient's skin or other biological tissue; (c) pumping the carrier fluid from the pump through or adjacent to the at least one opening of each of the reservoirs; (d) combining in the flow channel the carrier fluid from the pump with the drug from at least one of the reservoirs to form a fluidized drug; and (e) pumping the fluidized drug through the needle and into the patient. In a certain embodiment, the pump patch comprises a plurality of microneedles. In one embodiment, the pump patch further includes a plurality of discrete reservoir caps, each cap closing the at least one reservoir opening of each reservoir. The pump patch may further include a controller and a power source for actively disintegrating the plurality of reservoir caps to initiate the combining of the drug with the carrier fluid in the flow channel.

In another aspect, a device is provided for use in dialysis. In one embodiment, the device includes (i) a non-disposable module which comprises a pump or pressure generator; (ii) a disposable cassette operably connected to the pump or pressure generator, wherein the cassette includes a plurality of discrete reservoirs, each having at least one reservoir opening, reservoir contents located in the reservoirs, which reservoir contents comprise a drug, a sensor or sensor component, or a combination thereof, and a plurality of discrete reservoir caps, each cap closing the at least one reservoir opening of each reservoir; and (iii) power and control electronics for actively and selectively disintegrating the reservoir caps to expose the reservoir contents to a physiological fluid, a dialysate, or a combination thereof. The power and control electronics may be incorporated into the device in either or both of the disposable and non-disposable modules. In one embodiment, the reservoir contents includes a sensor or sensor component which can measure or monitor temperature, pH, salt concentration, metabolites, waste products, and/or blood gases of the blood or peritoneal fluid of a dialysis patient while the patient is be dialyzed. In one embodiment, the reservoir contents comprises a sensor or sensor component which can measure or monitor blood coagulation by measuring the level of one or more anti-coagulants, blood viscosity, clotting time, or a combination thereof. In still another embodiment, the reservoir contents comprises an anti-coagulant or other drug for release.

In yet another aspect, a fluidics connection device is provided. In one embodiment, the device includes a first substrate portion which comprises a sealing surface, an opposing surface, and at least one microfluidic via therethrough; a nipple connector which comprises sealing surface and at least one fluid aperture therethrough; and a compression cold weld seal which attaches the sealing surface of the first substrate portion to the sealing surface of the nipple connector, such that the microfluidic via is aligned in fluid communication with the fluid aperture. In a certain embodiment, the devices has a plurality of microfluidic vias and a plurality of corresponding fluid apertures, wherein the interface of each via with its corresponding fluid aperture is surrounded by a separate compression cold weld seal. In one embodiment, the compression cold weld seal comprises at least one ridge feature on one of the sealing surfaces and at least one groove in the other of the sealing surfaces. In one embodiment, the fluidics connection device further includes a second substrate portion attached by at least one compression cold weld seal to the opposing surface of the first substrate portion, wherein the second substrate comprises a second microfluidic via and/or microfluidic channel.

### BRIEF DESCRIPTION OF THE DRAWINGS

FIGS. 1A-B are cross-sectional views of a schematic representation of a prior art pressurized reservoir pump which is operated by a gas generation mechanism.

FIGS. 2A-B are cross-sectional views illustrating one embodiment of a transdermal drug delivery patch having an elastic bladder, a reservoir array, and a microneedle.

FIGS. 3A-B are cross-sectional views illustrating one embodiment of a transdermal pump patch comprising an array of microneedles and a reusable module containing control electronics and a power source.

FIGS. 4A-C are process flow diagrams illustrating some of the possible design configurations of the pump devices and systems described herein. FIG. 4A shows one embodiment of an active pumping system with active drug reservoirs. FIG. 4B shows one embodiment of a passive pumping device with active drug reservoirs. FIG. 4C shows one embodiment of a passive pumping device with a passive drug reservoir array.

FIG. 5 is a cross-sectional view of one embodiment of a transdermal pump patch which includes a syringe pump with reservoirs having active reservoir caps with opposing passively rupturable reservoir caps.

FIG. 6 is a cross-sectional view of one embodiment of a transdermal pump patch which includes a pressurized reservoir pump with reservoirs having active reservoir caps with opposing passively rupturable reservoir caps.

FIGS. 7A-C are cross-sectional views illustrating operation of another embodiment of a transdermal pump patch which incorporates a pressurized reservoir pump and a passive drug reservoir array.

FIGS. 8A-B are cross-sectional views illustrating operation of another embodiment of a transdermal pump patch that has a pressurized reservoir pump and a source for generating pressure to push drug out of a reservoir array after active reservoir caps have been removed.

FIGS. 9A-B are perspective views of one embodiment of a diffusion mixer which comprises two substrates designed with mating ridge and grooves which can be bonded together using compression cold welding. FIG. 9B is an exploded view with substrate 300 shown in a transparent view.

FIGS. 10A-B are cross-sectional views (FIG. 10A exploded view and FIG. 10B assembled view) of one embodiment of a fluidics device coupling a macroscale nipple connector to substrates which comprise microscale fluidic channels, designed with mating ridge and grooves which can be bonded together using compression cold welding.

FIG. 11 is a cross-sectional view of one embodiment of fluidic interfacing device for coupling together macroscale nipple connectors with a plurality of closely spaced microscale fluidic vias, designed with mating ridge and grooves which can be bonded together using compression cold welding.

FIG. 12 is a cross-sectional view of one embodiment of device that includes both electrical and fluidic connections which include mating ridge and grooves which can be bonded together using compression cold welding.

FIG. 13 is a partial cross-sectional view of an embodiment of a reservoir pump device.

### DESCRIPTION OF THE PREFERRED EMBODIMENTS

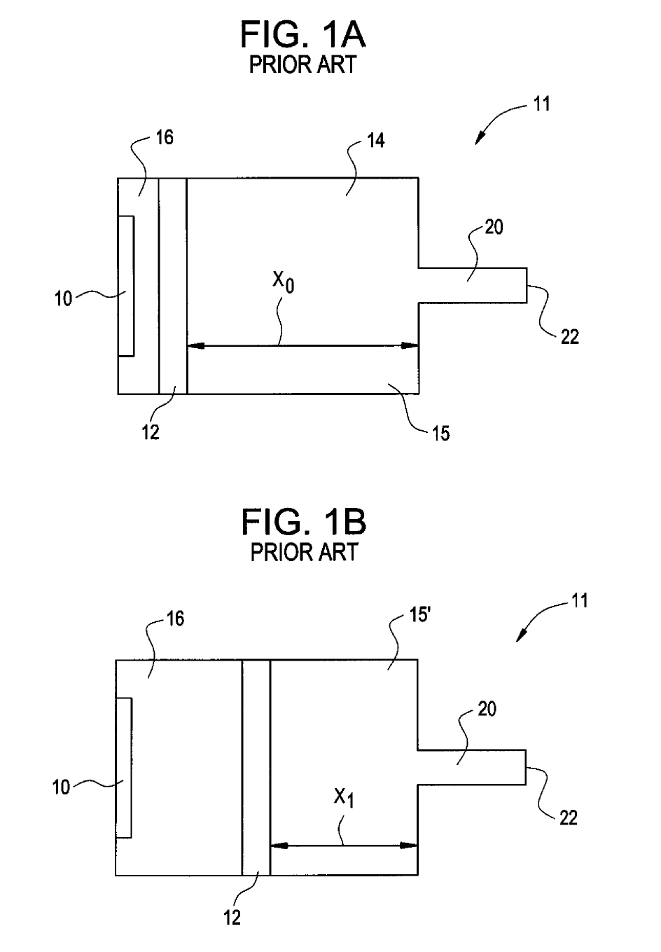

The foregoing problem with conventional pressurized reservoir pump systems is illustrated in FIGS. 1A-B. The figures show a conventional pressurized reservoir pump device 11 which operates by a gas generation mechanism. Gas generated by a gas generating means 10 (from electrolysis of water, an electrochemical reaction, or a chemical reaction) enters the pressurization chamber 16 and moves piston 12. The change in piston position (x₀-x₁) is proportional to the volume of the drug solution 15 delivered through orifice 22 from the reservoir 14 and the flow tube 20. Since the dose of the drug is directly proportional to the volume of the drug solution ejected from the orifice, any variation in pressure from the generated gas caused by changes in temperature, liquid viscosity, or other factors may result in inaccurate drug dosing.

To address this and other problems, improved multi-reservoir pump devices are provided. The devices include reservoirs for storing drug or other contents in need of temporary protection and a pumping means for delivering a carrier fluid and means for selectively contacting/combining the reservoir contents and carrier fluid to form a fluidized drug. As used herein, the term “fluidized drug” includes, but is not limited to, drug solutions (drug dissolved in a liquid), drug suspensions (drug particles suspended in a liquid), and drug emulsions.

The present devices may be adapted to pump materials to, into, or through a variety of biological tissues. As used herein, the term “biological tissue” includes essentially any cells, tissue, or organs, including the skin or parts thereof, mucosal tissues, vascular tissues, lymphatic vessels, ocular tissues (e.g., cornea, conjunctiva, sclera, choroid, retina), and cell membranes. The biological tissue can be in humans or other types of animals, particularly mammals. Human skin is the biological tissue of particular use and interest with the present devices and methods.

In one embodiment, the reservoir device is in the form of a transdermal patch device that includes a needle or other means for delivering the fluidized drug into or through a patient's skin. In another embodiment, the reservoir device is in the form of a dialysis cassette, or cartridge. The reservoir device may be integral to the transdermal pump or other pump, or the reservoir device may be a cartridge or cassette that can be plugged into another device that includes a pump.

By storing the molecules to be released separately from the carrier fluid (e.g., a diluent) to be pumped, one is able to store unstable or sensitive molecules, as well as store molecules in any essentially any useful form. For example, certain drugs, such as proteins, may advantageously be stored in a lyophilized form for increased shelf-life of the molecules and thus devices containing the molecules have a longer shelf life. The present devices provide the ability to store solids and gels, allowing one to use/deliver drugs/drug forms that could not readily be delivered with a conventional pump.

Separation of the carrier fluid and the molecules to be released also may permit reuse of the pumping mechanism, the carrier fluid source, a control electronics system, and/or a power source, while the reservoir array is designed to be disposable. Specifically, different reservoir devices may be used with one generic pump platform. This feature also allows for the incorporation of safety features such as barcodes on the reservoir devices, radio frequency identification (RFID) tag connectors, interlocking shapes on the reservoir device and the pump platform, or patterns of the electrical connectors. In addition, more than one type of molecule or drug may be provided in each device, since each reservoir may contain different contents. Thus, a single carrier fluid reservoir may be used to deliver multiple drugs from one device.

The multi-reservoir pump devices are capable of storing concentrated drug doses for release because the devices reconstitute (e.g., dissolve or suspend) the drug in the carrier fluid. The multi-reservoir pump devices are also easily miniaturized because the drugs are stored in concentrated doses having small volumes. Thus, in a preferred embodiment, the reservoir array may comprise an array of microreservoirs.

Accuracy of the dose of drug or molecules delivered is also improved, because the dose can be determined by the mass of drug or molecules contained within the reservoirs and not by the volume or flow rate of a drug/diluent fluid (e.g., drug solution or suspension). The device can deliver a complete dose without requiring an excessively large volume of carrier fluid. The flow tube, particularly the region(s) contacting the reservoirs, is designed to obtain the desired drug concentration profile, considering factors such as the flow rate of the carrier fluid, the dissolution rate of the drug, and dead volumes. For instance, the flow tube may serve as or contain a mixing means (e.g., a static mixer) where agitation or mixing is required or useful to dissolve or suspend the drug in the carrier fluid. The contents of the reservoir array comprise the drug dosages, and in use the reservoirs of the array are emptied. Advantageously, because the dosage may be determined by the mass of the molecules contained in the reservoir array, the dosage delivered by the multi-reservoir pump devices are essentially unaffected by variable reservoir pressurization.

The present devices provide that the entire drug in a reservoir is completely transferred into the carrier fluid and thereby delivered to the patient. Thus, the exact drug concentration of drug in the carrier fluid is not critical to proper operation of the device, so long as the flow of carrier fluid is above a minimum threshold to get complete release/delivery of the drug over a specified period to achieve the proper dose. The flow tube and carrier fluid reservoir pressure preferably will be designed to provide the minimum flow to achieve the proper dose. In addition, drug waste is reduced because substantially complete delivery of the drug is achieved.

As used herein, the terms “comprise,” “comprising,” “include,” and “including” are intended to be open, non-limiting terms, unless the contrary is expressly indicated.

The Device

The multi-reservoir pump device includes one or more reservoir devices. A typical reservoir device may include a substrate, reservoirs, and reservoir caps. The reservoir device is integrated with, or attached to, an apparatus providing for the active and/or passive release of molecules into a carrier fluid provided in the apparatus. The reservoir device alternatively or additionally may house one or more sensors.

In one aspect, the device comprises a substrate; a plurality of discrete reservoirs in the substrate; one or more pharmaceutical agents stored in the reservoirs; discrete reservoir caps that prevent the one or more pharmaceutical agents from passing out from the reservoirs; control means for actuating release of the pharmaceutical agents from one or more of the reservoirs by disintegrating or permeabilizing the reservoir caps; a carrier fluid source; and a means for pumping the carrier fluid to flow and contact the released pharmaceutical agent.

In transdermal drug delivery applications, the device may also comprise a means for securing the device to the skin of the patient; and means for transdermally delivering the pharmaceutical agent and carrier fluid into/through the skin following release of the pharmaceutical agent from one or more of the reservoirs.

In another aspect, the device is used to deliver a diagnostic agent into or through the skin. For instance, the agent could be a small molecule metabolite reporter, used in glucose detecting.

In still another aspect, the device is not used to deliver substances for release, but to contain a plurality of sensors for selective exposure. For example, the device may be adapted to monitor critical analytes or compounds in a dialysis solution during dialysis. The device may also incorporate sensors and substances for release in the same device.

In a preferred embodiment, the multi-reservoir pump device is adapted for transdermal drug delivery. Transdermal drug delivery patches, or pump patches, are well tolerated and accepted by patients, enable home use instead of hospital/clinic use, and are smaller and less expensive than traditional externally worn mechanical pumps (e.g., a syringe pump). For transdermal drug delivery, the multi-reservoir pump device may include a device housing containing the multi-reservoir pump device. The device housing may be in the form of a patch to be applied to the patient's skin. In some embodiments, an adhesive may be used to affix the device housing, or patch, to a patient's skin. In addition, some embodiments of the transdermal patch pump device have a needle or needles which automatically deploy (i.e., not seen by patient), which could replace frequent (e.g., daily) injections or injectable depots which have a slow continuous release, thus decreasing injection site reactions.

In another preferred embodiment, the present reservoir devices are adapted for use in dialysis, including hemodialysis, peritoneal dialysis, liver dialysis (for the removal of lipophilic, albumin-bound substances such as bilirubin, bile acids, metabolites of aromatic amino acids, medium-chain fatty acids and cytokines), and hemofiltration. In a preferred embodiment, the pump or pressure generator is part of the non-disposable dialysis equipment and the reservoirs (containing the drug, other molecules, and/or sensors) are located in a disposable cassette. The dialysis cassette may be adapted to simply be plugged into a conventional dialysis unit that includes a fluid reservoir and a pumping means. In an alternative embodiment, the pump or pressure generator is also part of the disposable cassette, though this typically would be less desirable from a cost perspective. The multi-reservoir pump device may be disposed in, fabricated on, or integrated into dialysis cassettes such as the ones described in U.S. Pat. No. 5,989,423, which is incorporated herein by reference.

The reservoir array in the dialysis cassette (i.e., cartridge) may contain drug or other molecules for release into a dialysate, or directly into the patient's blood or peritoneal fluid. Release kinetics may be pre-programmed or actively controlled, e.g., by remote control or based on feedback from a biosensor. In one case, release of drug from reservoirs in the dialysis cassette may be based on information from one or more sensors also located in the dialysis cassette, e.g., in reservoirs of an array or in other locations such as the flow channels, ports, or manifolds. In some cases, the sensors may be “off the shelf” type sensors and may not be exposed to bodily fluids for more than a few hours, so the sensors may not need to be stored/protected in discrete, sealed reservoirs. Release may be into a dialysis solution (dialysate), the patient's blood or peritoneal fluid, or a combination thereof.

In a preferred embodiment, the reservoir array in the dialysis cassette includes sensors-which may or may not be located in the reservoirs, depending for example on the shelf life of the sensor. For example, it would be highly advantageous to measure or monitor certain electrolytes or salts (e.g., potassium, sodium, phosphate), metabolites (e.g. urea), waste products, and/or blood gases in the dialysis patient while the patient is be dialyzed. In one example, the sensor may be used to monitor blood coagulation by measuring the concentration of one or more anti-coagulants in the blood or by measuring blood viscosity or clotting time, or a combination thereof, using one or more sensors known in the art. See Srivastava, Davenport, and Bums, “Nanoliter viscometer for analyzing blood plasma and other liquid samples,” Analytical Chemistry, 77(2);383-92 (2005). Such technologies may be integrated/adapted for use in dialysis cartridges and used to measure viscosity of various bodily/physiological fluids, including blood plasma, whole blood, etc. The sensor may, for instance, detect levels of heparin, warfarin, or other anti-coagulants in the blood. In another case, the sensor is one for detecting temperature, pH, or the concentration of certain analytes or waste products (e.g., urea, potassium). Such sensor may be helpful for monitoring the progress of the dialysis or, alternatively, another property indicative of patient health not (directly) related to renal function. In the former case, the sensor may enable the dialysis process to be completed in less time, for example, by real-time monitoring the effluent waste content, which may negate the perceived need to continue dialysis beyond the actual level required.

In one case, the sensors are part of the disposable dialysis cassette and are designed to operate for only a few hours. By sealing these sensors in reservoirs, the sensors can be protected from the environment while on the shelf, and then can be controllably/selectively exposed to fluids (e.g., body fluids, dialysate) during the dialysis process. This may permit the use of sensor chemistries that would otherwise be useless, for example due to their limited stability or shelf-life (if not protected in sealed reservoirs. In one case, it may desirable to avoid exposing the sensor during a first dialysis cycle, and then to open the reservoir and expose the sensor in a subsequent cycle. In another case, it may be desirable to expose the sensor at a particular step of the dialysis procedure, so that it can properly “wet up” and reach steady state before being exposed to the fluid including the analyte of interest. By locating the drug reservoirs or sensors in the disposable cassette, one may utilize the power and control electronics of the non-disposable dialysis machine to control reservoir opening and/or to collect/process sensor data, thereby providing cost savings relative to having to provide power and control electronics onboard each disposable cassette.

### Illustrative Embodiments

For simplicity, only two, three, or four discrete reservoirs are shown in some Figures. However, it is understood that a reservoir array component or device may contain one or many more reservoirs. It is also understood that the number, geometry, and placement of each reservoir, reservoir cap, or other object (e.g., resistors (heaters), electrodes, or flow channels) in or near each reservoir can be modified for a particular application. It is envisioned that various pump means and reservoir activation means (active, passive, mechanical rupture, electrothermal ablation, etc.) can be used and combined in different device designs other than those illustrated in the Figures without undue experimentation. In the figures, like parts are given like numbers.

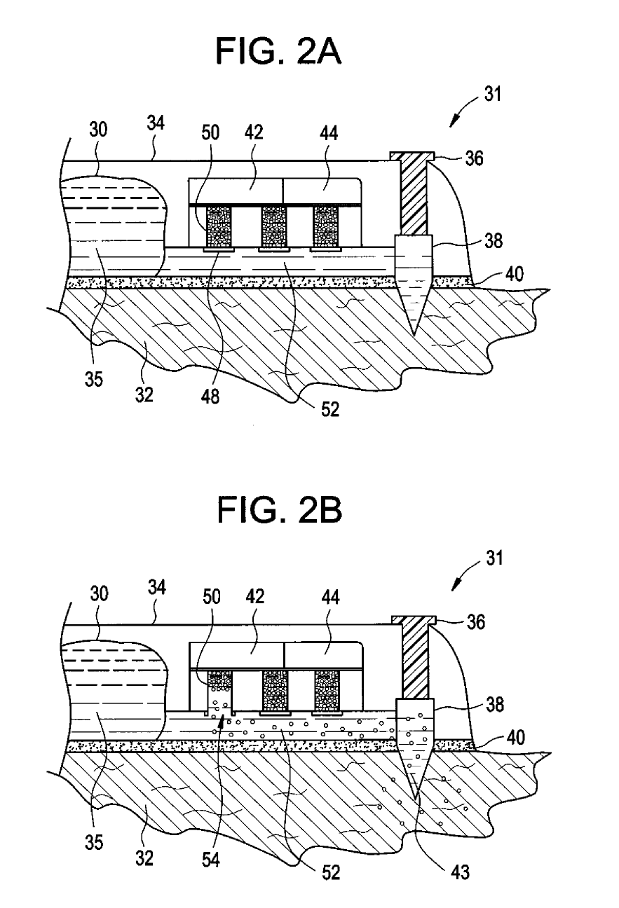

One embodiment of a transdermal drug delivery patch device 31 is shown in FIGS. 2A-B. The device includes a device housing 34 in which an elastic bladder 30 is disposed. The elastic bladder 30 contains a carrier fluid 35 and serves as a carrier fluid reservoir. The carrier fluid is pumped through a flow channel 52 by the pressure created by/within the elastic bladder. As shown, the carrier fluid is a liquid. (In an alternate embodiment the elastic bladder may be replaced by a gas generation mechanism and the fluid could be a gas.) The reservoirs 50 contain a drug formulation 33. Openings in the reservoirs are covered by reservoir caps 48 disposed within the flow channel 52. The patch device 31 also includes control electronics 42, a power source 44, and a microneedle 38 (or macroscale needle) for delivering the drug and the carrier fluid into a patient's skin 32. The microneedle 38 is provided with a plunger mechanism 36 for inserting the microneedle into the skin 32 following application of the patch to the skin 32. The patch device is affixed to the skin 32 by an adhesive layer 40. Deployment of the microneedle need not be seen by the patient.

Release of the drug formulation into the carrier fluid in the flow channel 52 is initiated by disintegration of the reservoir caps 48. FIG. 2B illustrates the opened reservoir 54 having its contents (i.e., drug formulation) released into the carrier fluid, combined with the carrier fluid to form a drug solution 43 and though the microneedle 38. The carrier fluid is caused to flow through the flow channel and through the microneedle 38 due to the pressure created in the elastic bladder 30. In preferred embodiments, the device provides that the flow of carrier fluid 35 is unidirectional through the flow channel 52, for example so that contamination of the carrier fluid reservoir is avoided. This could be accomplished for example by using a check valve and/or by ensuring a minimum flowrate.

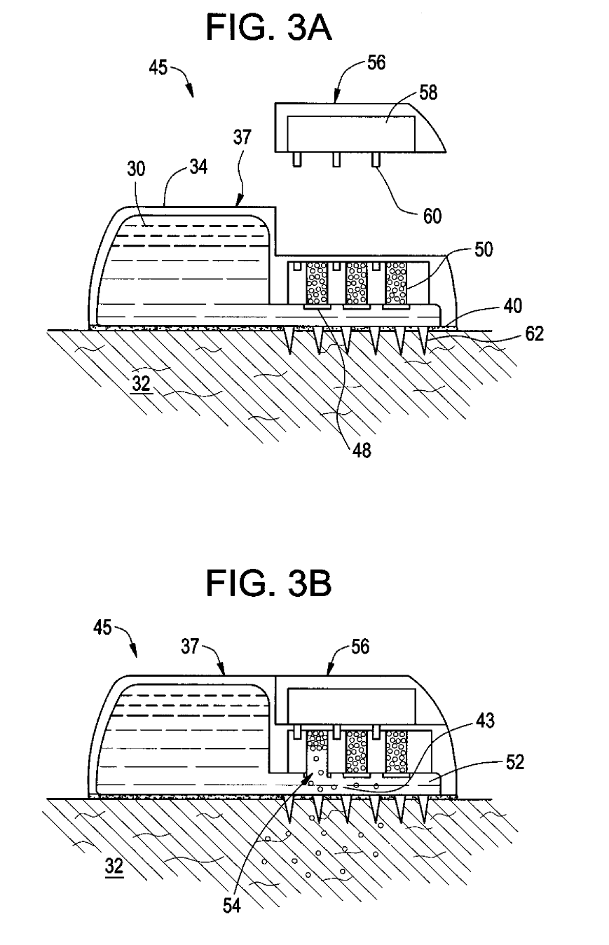

Another embodiment of a transdermal drug delivery patch device 45 is illustrated in FIGS. 3A-B. The device includes a reusable module 56 and a drug reservoir array module 37, which are releasably securable together. The reusable module 56 contains control electronics and a power source 58. Pins 60 electrically connect the reusable electronics and power source 58 to the drug reservoir array module 37. The drug reservoir array module 37 includes a microneedle array 62 to deliver drug solution 43 into a patient's skin 32.

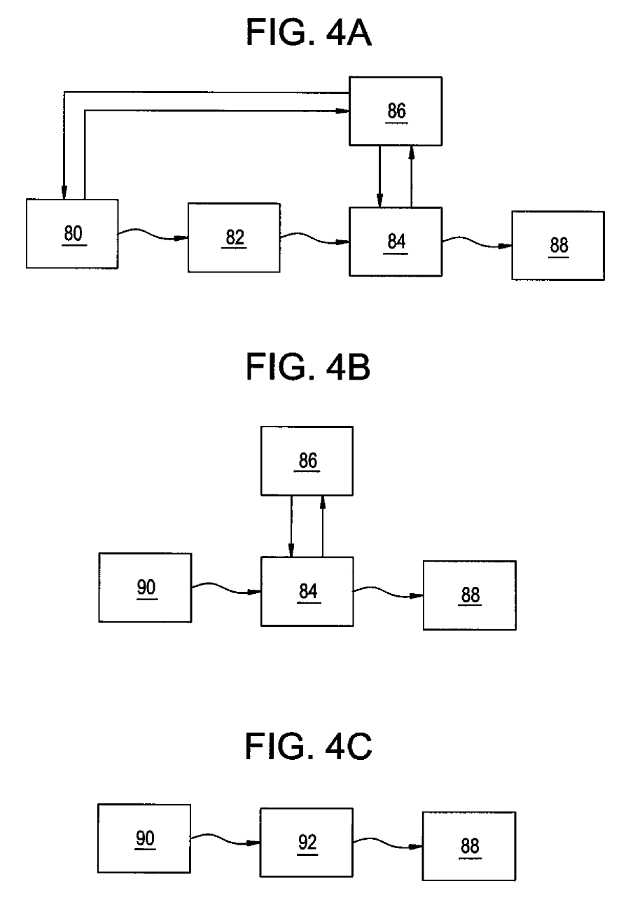

Various design configurations of the present medical devices are illustrated by block flow diagram in FIGS. 4A-C. FIG. 4A shows an active pumping system with active drug reservoirs. Block 80 is an active pumping mechanism (e.g., syringe pump, peristaltic pump) which is in fluid communication with a reservoir containing carrier fluid or diluent represented by block 82. Block 84 represents an active drug reservoir array in fluid communication with a carrier fluid source or diluent source. Block 86 represents control electronics and a power source, which communicates with the active pumping mechanism and active drug reservoir array to control their operation. Block 88 represents the drug delivery site (e.g., a patient). FIG. 4B is passive pumping device with active drug reservoirs. Block 90 represents a combined carrier fluid source and pumping mechanism (i.e., a pressurized reservoir). FIG. 4C is a passive pumping device with a passive drug reservoir array 92.

As used herein, the terms “active” in reference to pumps, pumping means, and pump systems includes devices that have mechanical moving parts, which typically require some kind of power source and control systems, such as with syringe pumps, peristaltic pumps, and the like.

The terms “passive pumping device” and pressurized reservoir pump“are typically used synonymously to refer to pumping means that do not have power source and control means. Elastic bladders and balloon systems, as well as osmotic pumps, are examples of “passive” pressure generation/fluid reservoir mechanisms.

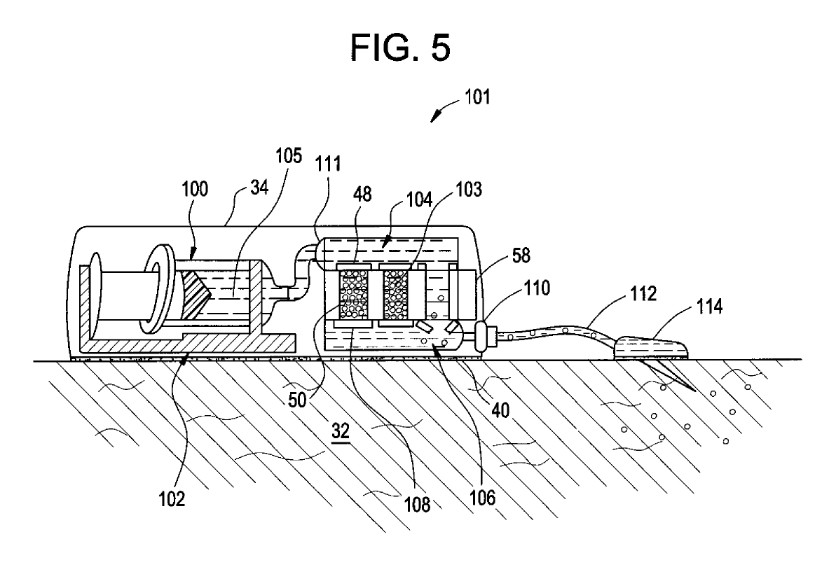

Another embodiment of a transdermal drug delivery patch device 101 is illustrated in FIG. 5. The patch device 101 includes a syringe pump 100 and a syringe pump drive mechanism 102, which are the active pumping mechanism and are contained in a housing 34. (In an alternate embodiment, the pump could be any other kind of active pump, such as a peristaltic pump.) Reservoirs 50 have actively disintegratable reservoir caps 48 covering openings at the top end of the reservoirs 50 and disintegratable reservoir caps 108 covering opposed reservoir openings at the bottom end of the reservoirs 50. Disintegration of reservoir caps 108 may be actively or passively disintegrated, as a matter of design choice. Reservoirs 50 are loaded with drug formulation 103. Operation of the device includes activation (disintegration) of one or more of reservoir caps 48, followed by pumping of carrier fluid 105 from carrier fluid reservoir, through check valve 111, into the upstream fluid manifold 104, and into the opened reservoirs. The pump 100 applies backpressure on the drug formulation contained within the reservoirs 50 to mechanically rupture the reservoir caps 108, or reservoir caps 108 can be actively disintegrated before or after activation of reservoir caps 48. The drug formulation 103 in the reservoir is then released into the downstream fluid manifold 106, where the drug formulation is dissolved into solution or suspended in the carrier fluid. (The upstream fluid manifold and the downstream fluid manifold may be structurally similar and may be referred to as “flow tubes.”) From the downstream manifold 106, the drug formulation and the carrier fluid pass through a check valve (e.g., a passive one-way valve) 110 to a catheter 112. The catheter 112 is in fluid communication with a subcutaneous needle insertion set 114 which delivers the drug formulation/carrier fluid into the skin 32. In contrast to the devices shown in FIGS. 2-3 where release of drug occurs by dissolution/diffusion from a reservoir and into a flowing carrier fluid, the device of FIG. 5 drives the carrier fluid through, and the drug out of, the reservoirs. That is, the carrier fluid is pushed against the drug formulation in a newly opened reservoir, allowing the simultaneous dissolution of the drug and the physical displacement of the drug from/drug solution out from the reservoir. A minimum flow of carrier fluid is preferably provided to prevent back flow of the drug solution into the upper manifold.

To operate embodiments where disintegration of reservoir caps 108 is intended to be passive, i.e., mechanically ruptured, sufficient pressure of carrier fluid will need to be generated for first and subsequent reservoir openings. As it may become increasingly difficult to generate a sufficient pressure differential across remaining scaled reservoirs once one or more reservoirs have been opened, the device may need to include a selective occlusion means to effectively re-seal an opened reservoirs once the drug has been flushed out. In one embodiment, this may be accomplished by using a hydrophilic expansion plug positioned in reservoir (e.g., at the outlet opening of the reservoir) which plug expands a short time after being exposed to an aqueous carrier fluid, thereby rendering closing off the used reservoir. Expansion plug materials and structures are known in the art, see, e.g., U.S. Pat. No. 4,781,683 to Wozniak, et al., which is incorporated herein by reference.

In an optional embodiment, the catheter may be separable from (i.e., it is removably attached to) the pump patch device, for replacement without having to replace the pump patch device at the same time. That is, the housed pump and reservoirs could remain on the skin for several days, while the subcutaneous needle, with or without the catheter, could be replaced and reinserted into a new location in the skin more frequently (e.g., every 3 to 7 days) in order to prevent infection. (In contrast, a conventional disposable pump would have to be entirely replaced every 3 to 7 days.) It is less expensive to replace the needle and catheter than it is to replace the pump mechanism, drug reservoirs, and fluid reservoirs. That is, the present device offers the benefit of a low-profiled, reasonably priced pump device, which is useful for a longer period of time before disposal/replacement is required, thereby making such a system more cost effective.

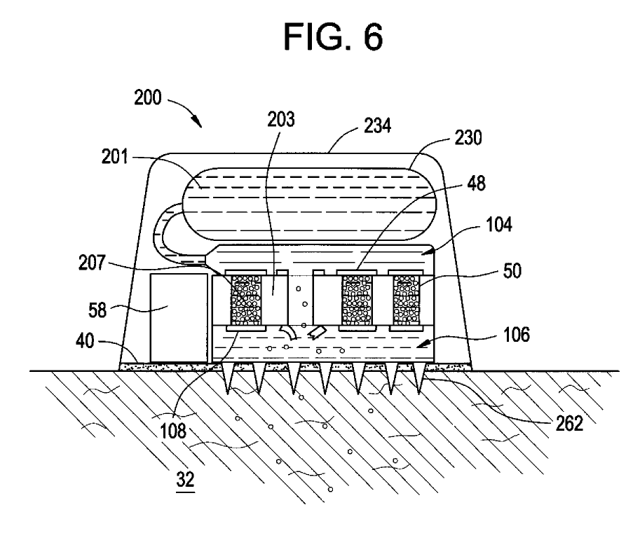

FIG. 6 illustrates another embodiment of a transdermal pump patch device 200. The device includes a device housing 234 and a pressurized reservoir pump, which comprises an elastic bladder 230 containing a carrier fluid 201, substrate 203 in which an array of discrete reservoirs 50 is disposed. Drug formulation 207 is stored in the reservoirs 50. The device 200 further includes active reservoir caps 48 and mechanically rupturable caps 108 respectively closing upper and opposed lower opening in reservoirs 50. In operation, carrier fluid is forced under pressure from bladder 230 into upper fluid manifold 104, and following activation of reservoir caps 48, travels through the reservoirs 50. The fluid pressure causes reservoir caps 108 to rupture, forcing the combination of drug formulation and carrier fluid into lower fluid manifold 106 and then through microneedles 262 and into the skin 32 of a patient. In an alternative embodiment, reservoir caps 108 may be active reservoir caps, opened before, simultaneously with, or after activation of reservoir caps 48.

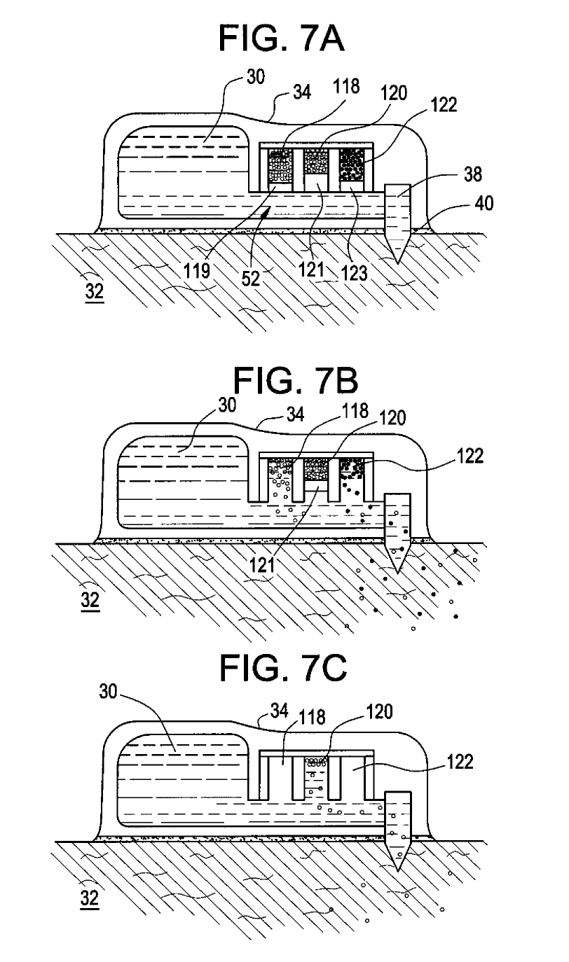

FIGS. 7A-C illustrate yet another embodiment of a transdermal pump patch incorporating a pressurized reservoir pump 30 and a passive drug reservoir array. The reservoirs are covered by passive release reservoir caps of varying thickness and contain different drugs. A first drug is contained in reservoir 118, which is covered by a (relatively) thin reservoir cap 119. The same drug is contained in reservoir 120, which is covered by a (relatively) thick passive reservoir cap 121. A second drug is contained in reservoir 122, which is covered by a thin passive reservoir cap 123. FIG. 7A shows the patch before any passive reservoirs have begun release. FIG. 7B shows release of the first drug from the reservoir 118 and the release of the second drug from the reservoir 122 after the thin caps have completely dissolved but the thick cap 121 is only partially dissolved. FIG. 7C shows release of the first drug from the thick cap reservoir 120 and two empty reservoirs 118, 122 that had thin caps. Alternatively, all the membranes may be of the same thickness, but of a different composition such that the caps dissolve at different rates. See, e.g., Grayson, et al. “Multi-pulse drug delivery from a resorbable polymeric microchip device”, Nature Materials, Vol. 2, November 2003.

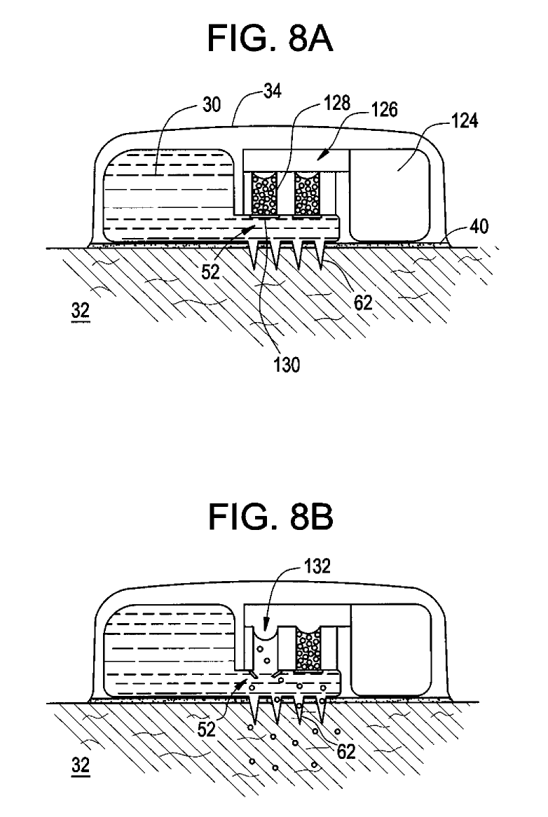

FIGS. 8A-B illustrate one embodiment of a transdermal pump patch that has a pressurized reservoir pump 30 for the carrier fluid and a separate source for generating pressure 124 to push the drug out of the reservoir array after the active reservoir caps are removed. The pressure generating source 124 creates pressure within a pressure manifold 126 having flexible membranes 132 contacting the contents of the reservoirs 128. As the active reservoir caps 130 on the opposite surface of the reservoir array are opened, the pressure in the pressure manifold 126 causes the flexible membranes to empty the contents of the reservoirs into carrier fluid in the flow tube 52 and the microneedles 62. FIG. 8A illustrates the device before the release of the drug from the reservoirs, and FIG. 8B illustrates the device after one reservoir cap has been opened and the drug has been released into the flow channel 52 and passes through the microneedles 62. If flexible membrane 132 is an elastic diaphragm, the relative pressure may be cycled to promote mixing in the reservoir, dissolution, and release of drug from the reservoir. Alternatively, synthetic jets could be used to promote mixing and release.

Gas/pressure generation may be produced by pyrotechnic means, for example, by detonating minute quantities of nitroglycerin using an electric current discharge. See also, U.S. Pat. No. 5,167,625 to Jacobsen et al. In other variations, the drug formulation may be displaced from the reservoirs using or adapting the means described in U.S. Patent Application Publications No. 2005/0055014 and No. 2004/0106914, both to Coppeta et al., which are incorporated herein by reference.

In still another embodiment, which is a variation of the embodiment shown in FIG. 6, though not illustrated, both reservoir caps 48 and reservoir caps 108 are activation (mechanically ruptured) by fluid pressure, such that no electronics are required. This passive patch system is operated by affixing the device to a patient's skin and then applying pressure (e.g., manually pressing) to a carrier fluid-filled elastic bladder (carrier fluid reservoir) to increase the pressure in an amount effect to cause the reservoir caps at both ends of the reservoir to fail, causing the carrier fluid to flow through the reservoirs and release the drug formulation therein. In order to only rupture a selected one or more reservoirs in the reservoir array, different reservoirs could be provided with reservoir caps that are formed of stronger materials or are thicker or both, in order to rupture require different but increased pressures (relative to the ones previously ruptured), or alternatively groups of reservoirs could be zoned for and in communication with different elastic bladders, each of which can be separately activated.

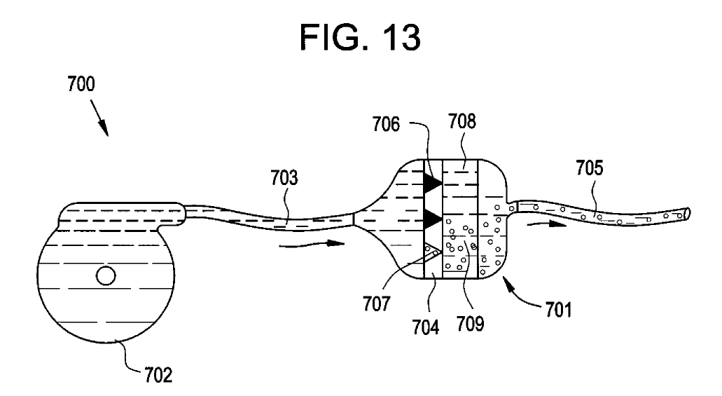

FIG. 13 illustrates an example of a pump and reservoir device 700 which includes a remote pump 702 which is in fluid communication with reservoir and mixing component 701 through flexible conduit 703. Reservoir and mixing component 701 includes substrate 704 with drug-containing reservoirs 706 arraying therein. Each reservoir has two opening on opposed sides of the substrate. Reservoirs caps (not shown) are provided over these openings and can be actively disintegrated to allow pumped carrier fluid to flow into and through the reservoirs and then into mixing space 708, as illustrated in opened reservoir 707. The fluidized drug 709 then can flow out of the device through discharge tube 705.

In variations of the foregoing embodiments, the release of molecules into the liquid carrier may be actively initiated by one of several mechanisms, which are detailed below in the section entitled Reservoir Caps and Control/Activation Means. In other variations, release of the reservoir content into the carrier fluid is passively controlled by one of several mechanisms, which are detailed below in the sections entitled Chemical Substances, Drugs, and Release-Controlling Materials (describing controlled release with the use of a passive release system). and Reservoir Caps and Control/Activation Means (describing controlled release/exposure with the use of passive reservoir caps).

In other embodiments, the present pump devices are tailored for operation with gaseous carrier fluids, for example, for use in inhalation (pulmonary or nasal) drug delivery. In one case, air is forced across reservoir openings (as in FIG. 3), or through reservoirs (as in FIG. 5) and drug formulation from the reservoir becomes entrained in the air before being directed to a patient's respiratory system. For instance, the liquid carrier fluid reservoir in FIG. 3 could be replaced with an air reservoir.

With the devices illustrated in FIGS. 2-8, it will typically be desirable for the devices to include means—such as a one-way, or check, valve—to ensure a unidirectional flow of the carrier fluid from the carrier fluid reservoir, so as to avoid contamination of the carrier fluid reservoir or pump parts with drug or physiological fluid.

In various embodiments of the devices described herein, the release kinetics of the drug may be tailored to achieve essentially any release profile needed. The devices may include various means for modifying the release profile. For example, the device includes a heater to increase the temperature of the carrier fluid to enhance dissolution and/or diffusion kinetics. In another example, the device may be designed to release the drug into the carrier fluid in the flow tube while the flow of carrier fluid is stopped, and then a bolus of the drug/carrier fluid is pumped into the patient once the dissolution of the drug in the carrier fluid is complete. In still another example, some reservoirs adjacent to drug-containing reservoir may be loaded with effluent modifiers to enhance dissolution of the drug formulation into the carrier fluid. The effluent modifier could be for example, ring compounds or solvents, such as non-polar solvents, DMSO, and the like, which increase the drug solubility. See also U.S. Patent Application Publications No. 2005/0267440 and No. 2006/0024358, which are incorporated herein by reference. This would allow the carrier fluid to remain relatively homogeneous, but change during a release event.

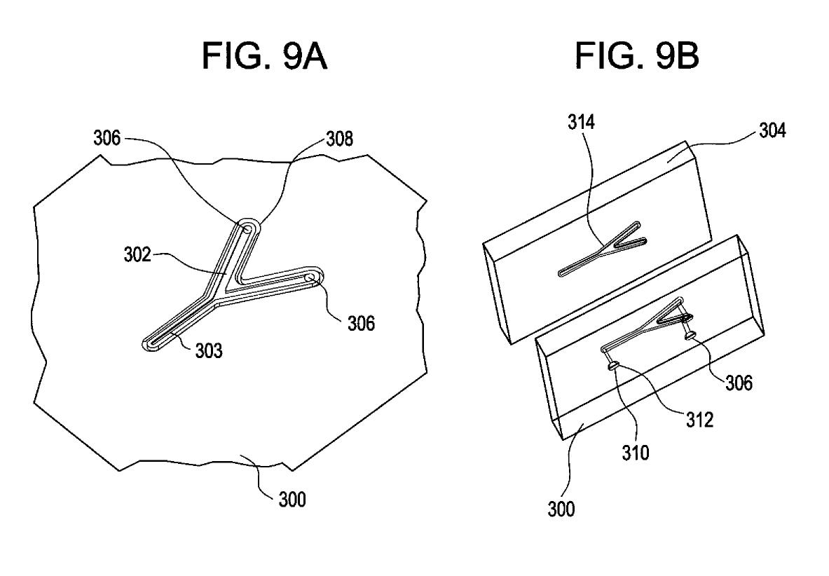

In another aspect, which is illustrated in FIGS. 9-12, devices and methods are provided using compression cold weld bonding techniques and structures to form fluid tight or hermetic connections between fluidic structures or devices, which will be particularly useful in the foregoing pump patch and dialysis type fluidic devices, which may combine macroscale carrier fluid flows and microscale reservoir devices and micron-scale flow channels. This compression cold welding methods and structures are described in U.S. Patent Application Publication No. 2006/0115323 to Coppeta et al., which is incorporated herein by reference. In one embodiment, the fluidic device includes a first substrate having a front side and a back side, and includes at least one first joint structure which comprises a first joining surface, which may be made of a first metal; a second substrate having at least one second joint structure which comprises a second joining surface, which may be made of a second metal; and a hermetic seal formed between and joining the first substrate and the second substrate. The hermetic seal may be made by compression cold welding the first joining surface to the second joining surface at one or more interfaces, preferably where the at least one second joint structure to locally deform and shear the joining surfaces at one or more interfaces in an amount effective to form a metal-to-metal bond between the first metal and second metal of the joining surfaces. The joining surfaces are joined together by a metal-to-metal bond formed without heat input, and the at least one first joint structure and the at least one second joint structure may comprise a tongue (ridge) and groove joint.

FIGS. 9A-B show a diffusion mixer, which may be used to mix two fluid streams together. The mixer may be formed by mating together substrate 300 with substrate 304. The substrates may be made of silicon, metals, or other material. For example, channels may be etched into silicon. Channels 302, 303 may be formed into one or both of the substrates at the interfacing surfaces of the substrates. The diffusion path length may be decreased by decreasing channel widths as the streams combine. For instance, as shown in FIG. 9A, flow channel 302 may have a width that is wider than the width of combined flow channel 303. Fluid ports 306, 310 through the ends of the channels act as inlets and outlets. Closed channels may be created by interlocking ridges and grooves in the interfacing surfaces of the substrates. Ridge 314 outlines the channels and mates with groove 308 in opposing substrate. The ends of fluidic ports 306 and 310 distal to the channel may also be provided with seal grooves for forming a fluid tight connection to other fluid transport components.

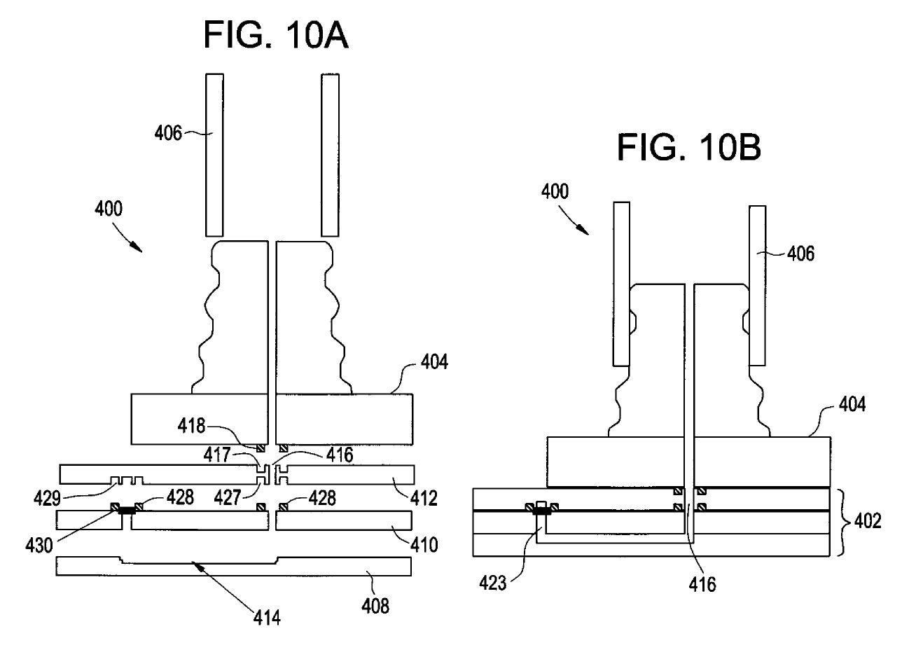

FIGS. 10A-B illustrate an example of a fluidics device 400 coupling a macroscale nipple connector 404 to substrate 402 which comprise microscale fluidic channels 414. Nipple connector 404 may be connected to Tygon tubing 406. Substrate 402 includes substrate portions 408, 410, and 412. Substrate portions 410 and 412 include fluidic via 416. Sealing ridges 418 and 428 engage into sealing grooves 417 and 427, respectively, to provide fluid tight connections for the fluidic vias and the interface of the substrate portion 412 to the nipple connector. Membrane 430 is disposed in port 423.

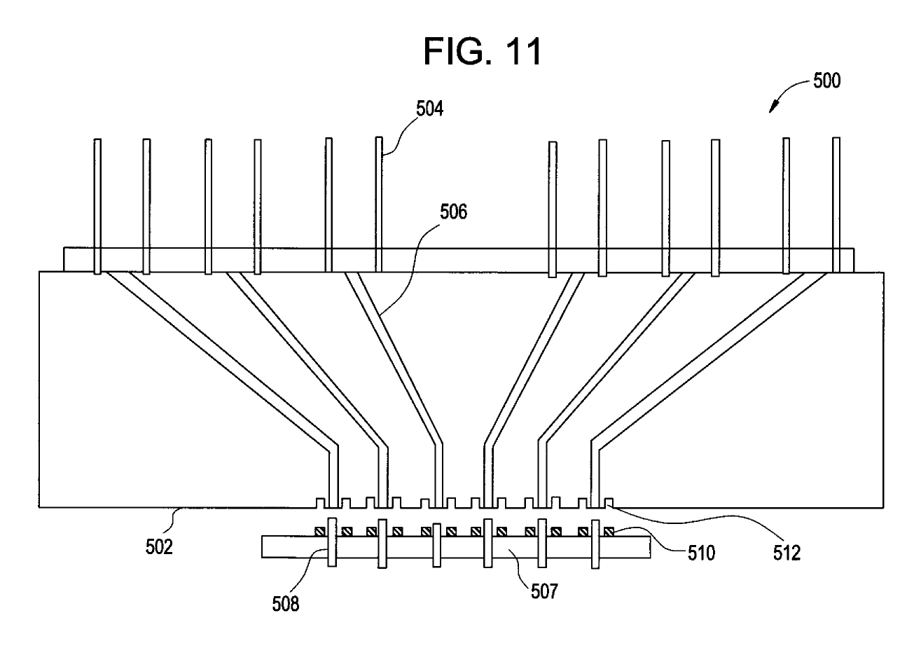

FIG. 11 shows an example of an interface device 500 for connecting multiple, closely spaced fluidic ports to a macro connector. The device 500 includes bulk connector 502 which includes fluidic channels 506 and nipple connectors 504. Substrate 507 includes fluidic vias 508 and is connected to bulk connector 502 sealing ridges 510 and sealing grooves 512. Other substrate portions which interface with substrate 507 are not shown.

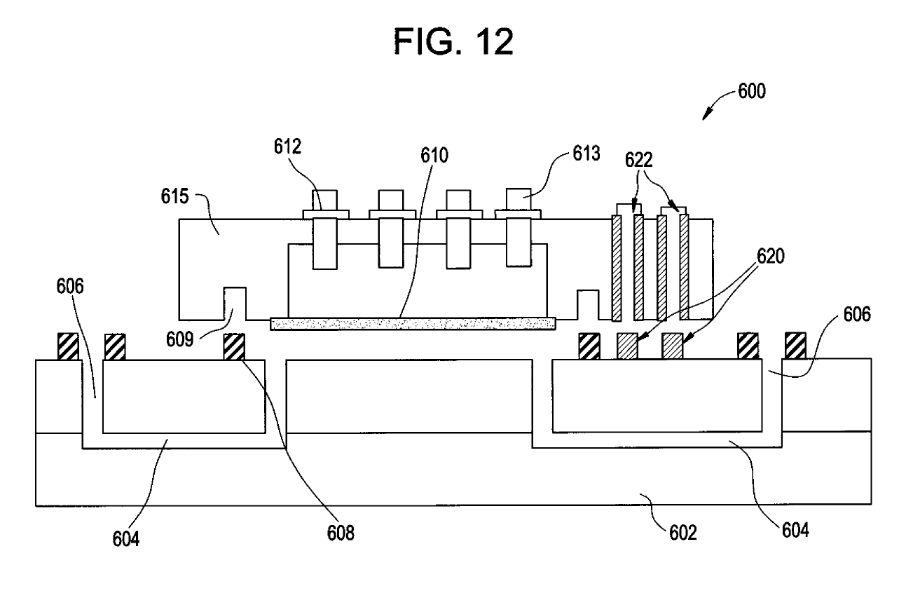

FIG. 12 shows an example of a device 600 for use in tissue capsule transport measurements. The device includes multiple electrical and fluidic connections. Base substrate 602 includes fluid channels 604 and fluid vias 606. Fluidic sealing ridges 608 mate with sealing groove 609 to connect base substrate to upper substrate 615 with semi-permeable membrane 610. The device includes electrical vias 622 which connect to electrical features 620. Fluidic connections 613 include membranes 612.

Reservoir Devices

The reservoir devices typically include a substrate having at least one reservoir, and more typically a plurality of reservoirs, containing reservoir contents to be selectively/controllably released or exposed. The reservoir devices in some embodiments further include one or more reservoir caps covering openings in the reservoirs. The reservoir caps may be designed and formed from a material which is selectively permeable to the molecules, which disintegrates/ruptures to release the molecules or a combination thereof. Active release systems may further include control circuitry and a power source. U.S. Pat. No. 5,797,898, No. 6,123,861, No. 6,491,666, No. 6,527,762, No. 6,551,838, No. 6,875,208, and No. 7,070,590 to Santini et al., are incorporated herein by reference.

The Substrate and Reservoirs

The substrate can be the structural body (e.g., part of a device) in which the reservoirs are formed, e.g., it contains the etched, machined, or molded reservoirs. In one embodiment, the containment device comprises a body portion, i.e., a substrate, that includes one or more reservoirs for containing reservoir contents sealed in a fluid tight or hermetic manner. As used herein, the term “hermetic” refers to a seal/containment effective to keep out helium, water vapor, and other gases. As used herein, the term “fluid tight” refers to a seal/containment which is not gas hermetic, but which is effective to keep out dissolved materials in a liquid phase (by excluding the liquid), for example, an analyte to be measured by a sensor sealed in a reservoir.

In preferred embodiments, the reservoirs are discrete, non-deformable, and disposed in an array across one or more surfaces (or areas thereof) of the device body. As used herein, the term “reservoir” means a well, a cavity, a recess, or a hole (which may be a through-hole, i.e., an aperture) suitable for storing, containing, and releasing/exposing a precise quantity of a material, such as a drug formulation, or a secondary device, or subcomponent. The randomly interconnected pores of a porous material are not reservoirs. In one embodiment the device includes a plurality of the reservoirs located in discrete positions across at least one surface of the body portion. In another embodiment, there is a single reservoir per each reservoir substrate portion; optionally two or more of these portions can be used together in a single device.

Reservoirs can be fabricated in a structural body portion using any suitable fabrication technique known in the art. Representative fabrication techniques include MEMS fabrication processes, microfabrication processes, or other micromachining processes, various drilling techniques (e.g., laser, mechanical, EDM, and ultrasonic drilling), and build-up or lamination techniques, such as LTCC (low temperature co-fired ceramics). The surface of the reservoir optionally can be treated or coated to alter one or more properties of the surface. Examples of such properties include hydrophilicity/hydrophobicity, wetting properties (surface energies, contact angles, etc.), surface roughness, electrical charge, release characteristics, and the like. MEMS methods, micromolding, micromachining, and microfabrication techniques known in the art can be used to fabricate the substrate/reservoirs from a variety of materials. Numerous other methods known in the art can also be used to form the reservoirs. See, for example, U.S. Pat. No. 6,123,861 and U.S. Pat. No. 6,808,522. Various polymer forming techniques known in the art also may be used, e.g., injection molding, thermocompression molding, extrusion, and the like.

In various embodiments, the body portion of the containment device comprises silicon, a metal, a ceramic, a glass, a polymer, or a combination thereof. Examples of suitable substrate materials include metals (e.g., titanium, tantalum, stainless steel, various other alloys such as cobalt-chrome), ceramics (e.g., alumina, silicon nitride), semiconductors (e.g., silicon), glasses (e.g., Pyrex™, BPSG), and degradable and non-degradable polymers. Where only fluid tightness is required, the substrate may be formed of a polymeric material, rather than a metal or ceramic which would typically be required for gas hermeticity. It is noted, however, that polymeric devices may be made gas hermetic, for example, if the material were a liquid crystal polymer of certain geometries or, alternatively, another polymer provided with a metal or ceramic coating.

In one embodiment, each reservoir is formed of (i.e., defined in) hermetic materials (e.g., metals, silicon, glasses, ceramics) and is hermetically sealed by a reservoir cap. Desirably, the substrate material is biocompatible and suitable for long-term implantation into a patient. In a preferred embodiment, the substrate is formed of one or more hermetic materials. The substrate, or portions thereof, may be coated, encapsulated, or otherwise contained in a hermetic biocompatible material (e.g., inert ceramics, titanium, and the like) before use. Non-hermetic materials may be completely coated with a layer of a hermetic material. For example, a polymeric substrate could have a thin metal coating. If the substrate material is not biocompatible, then it can be coated with, encapsulated, or otherwise contained in a biocompatible material, such as poly(ethylene glycol), polytetrafluoroethylene-like materials, diamond-like carbon, silicon carbide, inert ceramics, alumina, titanium, and the like, before use. In a preferred embodiment, the substrate is hermetic—that is, impermeable at least during the time of use of the reservoir device—to the molecules to be delivered and to surrounding gases or fluids (e.g., water, blood, electrolytes or other solutions).

The substrate may be formed into a range of shapes or shaped surfaces. It can, for example, have a planar or curved surface, which for example could be shaped to conform to an attachment surface, such as the skin. In various embodiments, the substrate or the containment device is in the form of a planar chip, a circular or ovoid disk, an elongated tube, a sphere, or a wire. The substrate may be flexible or rigid. In one embodiment, the reservoirs are discrete, non-deformable, and disposed in an array across one or more surfaces (or areas thereof) of an implantable medical device.

The substrate may consist of only one material, or may be a composite or multi-laminate material, that is, composed of several layers of the same or different substrate materials that are bonded together. Substrate portions can be, for example, silicon or another micromachined substrate or combination of micromachined substrates such as silicon and glass, e.g., as described in U.S. Patent Application Publication 2005/0149000 or U.S. Pat. No. 6,527,762. Representative examples of glasses include aluminosilicate glass, borosilicate glass, crystal glasses, etc. In another embodiment, the substrate comprises multiple silicon wafers bonded together. In yet another embodiment, the substrate comprises a low-temperature co-fired ceramic (LTCC) or other ceramic such as alumina. In one embodiment, the body portion is the support for a microchip device. In one example, this substrate is formed of silicon.

Total substrate thickness and reservoir volume can be increased by bonding or attaching wafers or layers of substrate materials together. The device thickness may affect the volume of each reservoir and/or may affect the maximum number of reservoirs that can be incorporated onto a substrate. The size and number of substrates and reservoirs can be selected to accommodate the quantity and volume of reservoir contents needed for a particular application, manufacturing limitations, and/or total device size limitations to be suitable for implantation into or onto a patient.

The substrate may have one, two, three or more discrete reservoirs. In various embodiments, tens, hundreds, or thousands of reservoirs may be arrayed across/in the substrate. For instance, one embodiment of an implantable drug delivery device may include an array of between 100 and 750 discrete reservoirs, where each reservoir contains a single dose of a drug for release. In one embodiment for sensing, the number of reservoirs in the device may be determined by the operational life of the individual sensors.

Each reservoir may have one opening or two or more openings, which are sealed with a reservoir cap. The two or more openings may be opposed from one another on distal surfaces of the substrate or may be adjacent to one another on the same surface of the substrate. In certain alternative embodiments, the reservoirs have no reservoir caps, for example, in some cases where the reservoir contents comprises a release system for passive controlled release of one or more chemical molecules (e.g., drug molecules heterogeneously or homogeneously dispersed in a matrix material). In one case where a reservoir has two opposed openings, each of the openings may be sealed with a discrete reservoir cap, or alternatively, one of the openings may be sealed with a reservoir cap and the other opening may be sealed by a material that is intended to be permanent, i.e., it is designed not to be removed, degraded, permeabilized, or disintegrated during operation of the device.

In one embodiment, the reservoirs are microreservoirs. The “microreservoir” is a reservoir suitable for storing and releasing/exposing a microquantity of material, such as a drug formulation. In one embodiment, the microreservoir has a volume equal to or less than 500 μL (e.g., less than 250 μL, less than 100 μL, less than 50 μL, less than 25 μL, less than 10 μL, etc.) and greater than about 1 nL (e.g., greater than 5 nL, greater than 10 nL, greater than about 25 nL, greater than about 50 nL, greater than about 1 μL, etc.). The term “microquantity” refers to volumes from 1 nL up to 500 μL. In one embodiment, the microquantity is between 1 nL and 1 μL. In another embodiment, the microquantity is between 10 nL and 500 nL. In still another embodiment, the microquantity is between about 1 μL and 500 μL. The shape and dimensions of the microreservoir can be selected to maximize or minimize contact area between the drug material (or sensor or other reservoir contents) and the surrounding surface of the microreservoir. Reservoir volumes less than 1 nL are envisioned and may be desirable with certain devices.

In one embodiment, the reservoir is formed in a 200-micron thick substrate and has dimensions of 1.5 mm by 0.83 mm, for a volume of about 250 nL, not counting the volume that would be taken up by the support structures, which may be about 20 to about 50 microns thick.

In other embodiments, the reservoirs may be macroreservoirs. The “macroreservoir” is a reservoir suitable for storing and releasing/exposing a quantity of material larger than a microquantity. In one embodiment, the macroreservoir has a volume greater than 500 μL (e.g., greater than 600 μL, greater than 750 μL, greater than 900 μL, greater than 1 mL, etc.) and less than 5 mL (e.g., less than 4 μL, less than 3 mL, less than 2 mL, less than 1 mL, etc.).

Unless explicitly indicated to be limited to either micro- or macro-scale volumes/quantities, the term “reservoir” is intended to encompass both.

The substrate may further include reservoir cap support structures as described in U.S. Patent Application Publications No. 2006/0057737 and No. 2005/0143715 to Santini Jr., et al., which are incorporated herein by reference. Reservoir cap supports can comprise substrate material, structural material, or coating material, or combinations thereof. Reservoir cap supports comprising substrate material may be formed in the same step as the reservoirs. The MEMS methods, microfabrication, micromolding, and micromachining techniques mentioned above may be used to fabricate the substrate/reservoirs, as well as reservoir cap supports, from a variety of substrate materials. Reservoir cap supports comprising structural material may also be formed by deposition techniques onto the substrate and then MEMS methods, microfabrication, micromolding, and micromachining techniques. Reservoir cap supports formed from coating material may be formed using known coating processes and tape masking, shadow masking, selective laser removal techniques, photolithography, lift off, or other selective methods.

A reservoir may have several reservoir cap supports in various configurations over its reservoir contents. For example, one reservoir cap support may span from one side of the reservoir to the opposite side; another reservoir cap support may cross the first reservoir cap support and span the two other sides of the reservoir. In such an example, four reservoir caps could be supported over the reservoir. In one embodiment for a sensor application (e.g., a glucose sensor), the reservoir (of a device, which may include only one reservoir or which may include two or more reservoirs) has two, three, or more reservoir openings and corresponding reservoir caps. The dimensions and geometry of the support structure can be varied depending upon the particular requirements of a specific application. For instance, the thickness, width, and cross-sectional shape (e.g., square, rectangular, triangular) of the support structures may be tailored for particular drug release kinetics for a certain drug formulation or implantation site, etc.

Reservoir Contents

The reservoir contents may be essentially any object or material that needs to be stored and isolated (e.g., protected from) the environment outside of the reservoir until a selected time point when its release or exposure is desired. In various embodiments, the reservoir contents may include a quantity of drug or other chemical substance, a secondary device, or a combination thereof.

Following reservoir activation (i.e., opening), the reservoir contents become exposed to the environment outside of the reservoir. The contents may be released from the reservoir or may be retained (e.g., immobilized) within the reservoir, depending upon the particular reservoir contents and application. For example, a catalyst or sensor may not require release from the reservoir; rather their intended function, e.g., catalysis or sensing, can occur upon exposure of the reservoir contents to the environment outside of the reservoir after opening of the reservoir cap—and typically following ingress of one or more reactants or ingress of an analyte of interest. In an alternative case, the catalyst molecules or sensing component may be released from the opened reservoir, as would be typical when the reservoir contents comprises drug molecules, in order to exert a therapeutic effect on a patient. However, the drug molecules may be retained within the reservoirs for certain in vitro applications, such as drug screening activities like high-throughput screening or screening of molecule activity or stability when exposed to various chemicals, environmental conditions (e.g., pH), genetic materials, biowarfare agents, bacteria, viruses, or formulations.

Chemical Substances, Drugs, and Release-Controlling Materials

The reservoir contents can include essentially any natural or synthetic, organic or inorganic material, or mixtures thereof These substances may be stored in the reservoirs in essentially any form, such as a pure solid or liquid, a gel or hydrogel, a solution, an emulsion, a slurry, or a suspension. A particular substance of interest (e.g., the active ingredient) may be mixed with other materials to control the rate and/or time of release from an opened reservoir or enhance the stability, solubility, or complete release of the substance of interest. In various embodiments, the reservoir contents may be in the form of solid mixtures, including amorphous and crystalline mixed powders, monolithic solid mixtures, lyophilized powders, and solid interpenetrating networks. See, e.g., U.S. Patent Application Publications No. 2004/0247671 to Prescott et al. and No. 2004/0043042 to Johnson et al., which are incorporated herein by reference. In other embodiments, the reservoir contents are in a liquid-comprising form, such as solutions, emulsions, colloidal suspensions, slurries, or gel mixtures such as hydrogels.

In a preferred embodiment, the reservoir contents may include or consist essentially of one or more drug formulations. The drug formulation is a composition that comprises a drug. As used herein, the term “drug” includes any therapeutic or prophylactic agent (e.g., an active pharmaceutical ingredient or API) as known in the art. In one preferred embodiment, the drug is disposed in the reservoirs in a solid form, particularly for purposes of maintaining or extending the stability of the drug over a commercially and medically useful time, e.g., during storage in a drug delivery device until the drug needs to be administered. The solid drug formulation may be loaded into the reservoirs in a solid form or while in a liquid form which is subsequently solidified/precipitated using processes such as drying or lyophilization. The solid drug matrix may be in pure form or in the form of solid particles of another material in which the drug is contained, suspended, or dispersed.

The drug can comprise small molecules, large (i.e., macro-) molecules, or a combination thereof. In various embodiments, the drug can be selected from amino acids, vaccines, antiviral agents, gene delivery vectors, interleukin inhibitors, immunomodulators, neurotropic factors, neuroprotective agents, antineoplastic agents, chemotherapeutic agents (e.g., paclitaxel, vincristine, ifosfamide, dacttinomycin, doxorubicin, cyclophosphamide, fluorouracil, carmustine, and the like), growth factors (e.g., fibroblast growth factors, platelet-derived growth factors, insulin-like growth factors, epidermal growth factors, transforming growth factors, cartilage-inducing factors, osteoid-inducing factors, osteogenin and other bone growth factors, and collagen growth factors), polysaccharides, anticoagulants and/or antiplatlet drugs (e.g., low molecular weight heparin, other heparins, aspirin, clopidogrel, lepirudin, fondaparinux, warfarins, dicumarol, pentasaccharides, etc.), antibodies, antibiotics (e.g., immunosuppressants), anti-microbials, analgesic agents (such as opoids and NSAIDS), anesthetics (e.g., ketoamine, bupivacaine and ropivacaine), anti-proliferatives, anti-inflammatories, angiogenic or anti-angiogenic molecules, and vitamins. In one embodiment, the large molecule drug is a protein or a peptide. Examples of suitable types of proteins include glycoproteins, enzymes (e.g., proteolytic enzymes), hormones or other analogs (e.g., luteinizing hormone-releasing hormone, steroids, corticosteroids, growth factors), antibodies (e.g., anti-VEGF antibodies, tumor necrosis factor inhibitors), bisphosphonates (e.g., pamidronate, clodronate, zoledronic acid, and ibandronic acid), tramadol, dexamethasone, cytokines (e.g., α-, β-, or γ-interferons), interleukins (e.g., IL-2, IL-10), diabetes/obesity-related therapeutics (e.g., insulin, exenatide, PYY, GLP-1 and its analogs). Any form of insulin, including short acting, long acting, etc. may be suitable for use with the present reservoir devices. The drug may be a gonadotropin-releasing (LHRH) hormone analog, such as leuprolide. The drug may be a parathyroid hormone, such as a human parathyroid hormone or its analogs, e.g., HPTH(1-84), hPTH(1-34), or hPTH(1-31). The drug may be selected from nucleosides, nucleotides, and analogs and conjugates thereof. The drug may be a peptide with natriuretic activity, such as atrial natriuretic peptide (ANP), B-type (or brain) natriuretic peptide (BNP), C-type natriuretic peptide (CNP), or dendroaspis natriuretic peptide (DNP). In still other embodiments, the drug is selected from diuretics, vasodilators, inotropic agents, anti-arrhythmic agents, Ca⁺ channel blocking agents, anti-adrenergics/sympatholytics, and renin angiotensin system antagonists. The drug may be a vascular endothelial growth factor (VEGF) inhibitor, VEGF antibody, VEGF antibody fragment, or another anti-angiogenic agent. Examples include an aptamer, such as MACUGEN™ (Pfizer/Eyetech) (pegaptanib sodium)) or LUCENTIS™ (Genetech/Novartis) (rhuFab VEGF, or ranibizumab). The drug may be a prostaglandin, a prostacyclin, or another drug effective in the treatment of peripheral vascular disease. The drug may be an angiogenic agent, such as VEGF. The drug may be an anti-inflammatory agent, such as dexamethasone. In one embodiment, the multi-reservoir device includes both angiogenic agents and anti-inflammatory agents. The drug may be selected from antiparasitic agents, antiviral agents, cytotoxins or cell proliferation inhibiting agents.

The drug may be a self-propagating agent, such as a gene therapy agent or vector. The drug may be in the form of cells, e.g., adult stem cells.

The drug may be in an encapsulated form. For example, the drug can be provided in microspheres or liposomes for controlled release. The drug may be provided in nanoparticle form.

In a preferred embodiment, the reservoir contents may include an electrolyte (i.e., a salt for forming an aqueous solution of the salt), a metabolite, an anti-coagulant, erythropoietin, a red blood cell stimulating drug, or a molecule that may be depleted during dialysis. Such materials are known in the art.

The reservoirs in one device may include a single drug or a combination of two or more different drugs, and may further include one or more pharmaceutically acceptable carriers. Two or more transport enhancers, angiogenic agents, anti-inflammatory agents, or combinations thereof, may be stored together and released from the same one or more reservoirs or they may each be stored in and released from different reservoirs.

The reservoirs in one device may include a single drug in two or more different formulations, for example to provide different dosing profiles over time. For example, different therapeutic or prophylactic agents, or different doses, can be delivered from a single device, either from the same surface region or from different surface regions. In one embodiment, the quantity of therapeutic or prophylactic agent provided for release from at least a first of the reservoirs is different from the quantity of the therapeutic or prophylactic agent provided for release from at least a second of the reservoirs. In another embodiment, the time of release of one of the therapeutic or prophylactic agents from at least a first of the reservoirs is different from the time of release of the therapeutic or prophylactic agent from at least a second of the reservoirs. In one embodiment, a first therapeutic or prophylactic agent is in at least one of the reservoirs and a second therapeutic or prophylactic agent is in at least one other of the reservoirs, the first therapeutic or prophylactic agent and the second therapeutic or prophylactic agent being different in kind or dose.

The drug or other substances for release may be dispersed in a matrix material to control the kinetics of release. The matrix material may be polymeric, non-polymeric, hydrophobic, hydrophilic, lipophilic, amphiphilic, and the like. The matrix may be bioresorbable or non-bioresorbable. For example, this matrix material can be part of a “release system,” as described in U.S. Pat. No. 5,797,898, which is incorporated herein by reference. The degradation, dissolution, or diffusion properties of the matrix material can provide a means for controlling, for example, the rate at which the active ingredient is released from the reservoirs, the time at which release is initiated (e.g., following contact of the matrix material with a fluid outside of the reservoir), or both.

In one embodiment, release is initiated by degradation of the release system upon exposure to the carrier fluid. The chemical nature of the fluid, e.g., acid versus basic or polar versus non-polar, may cause the release system material, or matrix material thereof, to degrade or dissolve. The substance of interest will be released into the carrier fluid flowing adjacent to the reservoir opening as the matrix material is dissolved/degraded.

Particularly for drugs, the release system may include one or more pharmaceutical excipients. The release system may provide a temporally modulated release profile (e.g., pulsatile release) when time variation in plasma levels is desired or a more continuous or consistent release profile when a constant plasma level as needed to enhance a therapeutic effect, for example. Pulsatile release may be achieved from an individual reservoir, from a plurality of reservoirs, or a combination thereof For example, where each reservoir provides only a single pulse, multiple pulses (i.e., pulsatile release) are achieved by temporally staggering the single pulse release from each of several reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of molecules through it over an extended period. In addition, continuous release can be approximated by releasing several pulses of molecules in rapid succession (“digital” release).

In certain embodiments, the drug or other chemical substance is formulated in a sustained or controlled release formulation. Exemplary materials useful in preparing sustained release formulations include synthetic, biocompatible polymers known in the art. The polymer typically has a molecular weight greater than about 3000, preferably greater than about 10,000, and less than about 10 million, preferably less than about a million and more preferably less than about 200,000. Examples of polymers include poly-α-hydroxy acid esters, such as polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly(glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins; polypeptides and proteins, such as polylysine, polyglutamic acid, albumin; polyanhydrides; polyhydroxy alkonoates such as polyhydroxy valerate, polyhydroxy butyrate, and the like.

In one embodiment, the drug formulation within a reservoir comprises layers of drug and layers non-drug (i.e., matrix) material. After the active release mechanism has exposed the reservoir contents, the multiple layers provide multiple pulses of drug release due to intervening layers of non-drug. Such a strategy can be used to obtain complex release profiles. The technique could be used, for example, to deliver two different drugs that are incompatible with one another or otherwise should not be released at the same time. For instance, the layer structure could be non-drug/DrugA/non-drug/DrugB.

In another embodiment, the drug and matrix material can be provided in the reservoirs in a gradient form, where the concentration of the drug changes continuous with the depth in the reservoirs. For example, there may be a higher concentration of drug near one end (e.g., the end distal the opening of the reservoir) which decreases toward the other end. See, e.g., U.S. Patent Application Publication No. 2006/0147489, which is incorporated herein by reference.

The drug may be formulated with one or more excipients that facilitate transport through tissue capsules. Examples of such excipients include solvents such as dimethyl sulfoxide or collagen- or fibrin-degrading enzymes. See U.S. Patent Application Publication No. 2005/0267440 to Herman et al., which is incorporated herein by reference.

The drug may formulated with an excipient material that is useful for accelerating release, e.g., a water-swellable material that can aid in forcing the drug out of the reservoir, or otherwise provided in the reservoirs with components to effectuate more rapid release. See U.S. Patent Application Publication No. 2005/0055014 to Coppeta et al., which is incorporated herein by reference.

For in vitro applications, the chemical molecules stored in the reservoirs can be any of a wide range of molecules where the controlled release or exposure of a small (milligram to nanogram) amount of one or more types of molecules is required, for example, in the fields of analytic chemistry or medical diagnostics. The molecules may be effective as pH buffering agents, diagnostic reagents, and reagents in complex reactions such as the polymerase chain reaction or other nucleic acid amplification procedures. In various other embodiments, the molecules to be released are fragrances or scents, dyes or other coloring agents, sweeteners or other concentrated flavoring agents, or a variety of other compounds. In yet other embodiments, the reservoirs contain immobilized molecules. Examples include any chemical species which can be involved in a reaction, including reagents, catalysts (e.g., enzymes, metals, and zeolites), proteins (e.g., antibodies), nucleic acids, polysaccharides, cells, and polymers, as well as organic or inorganic molecules which can function as a diagnostic agent.

Release of the molecule from the reservoirs may be further controlled by the use of reservoir caps, including actively or passively reservoir disintegrated reservoir caps, or a combination of both actively and passively reservoir disintegrated reservoir caps, which are detailed below. For example, the reservoir cap can be removed by active means to expose a passive release system, or a multi-reservoir device can include one or more passive release reservoirs and one or more active release reservoirs.

Secondary Devices

As used herein, unless explicitly indicated otherwise, the term “secondary device” includes any device or a component thereof that can be located in a reservoir. Secondary devices are further described in U.S. Pat. No. 6,551,838 and in U.S. Patent Application Publication No. 2004/0248320, which are incorporated herein by reference.

In a preferred embodiment, the secondary device is a sensor or sensing component thereof. As used herein, a “sensing component” includes a component utilized in measuring or analyzing the presence, absence, or change in a chemical or ionic species, energy, or one or more physical properties (e.g., pH, temperature, pressure) at a site. Types of sensors include biosensors, chemical sensors, physical (e.g. mechanical) sensors, or optical sensors. Examples of sensing components include components utilized in measuring or analyzing the presence, absence, or change in a drug, chemical, or ionic species, energy (or light), or one or more physical properties (e.g., pH, pressure) at a site. The sensor may be a pressure sensor, as described in U.S. Pat. No. 6,221,024, No. 6,237,398, and No. 6,706,005, and U.S. Patent Application Publication No. 2004/0073137, which are incorporated herein by reference. The sensor may include a cantilever-type sensor, such as those used for chemical detection, as described in U.S. Patent Application Publication No. 2005/0005676, which is incorporated herein by reference. The secondary devices may be integral to the device or can be fabricated separately and added to the device. The device may be implantable in a patient (e.g., a human or other mammal). See, e.g., U.S. Patent Application Publications No. 2006/0076236 to Shah et al., and No. 2006/0025748 to Ye et al., which are incorporated herein by reference.

As used herein, the term “biosensor” includes sensing devices that transduce the chemical potential of an analyte of interest into an electrical signal (e.g., by converting a mechanical or thermal energy into an electrical signal), as well as electrodes that measure electrical signals directly or indirectly. For example, the biosensor may measure intrinsic electrical signals (EKG, EEG, or other neural signals), pressure, temperature, pH, or mechanical loads on tissue structures at various in vivo locations (e.g., strain gauges). In various embodiments, the biosensor may be one known in the art for use in measure an analyte selected from dissolved and total amounts of carbon dioxide, carbon monoxide, ammonia, dioxygen, ethanol, ionized calcium, sodium ion, potassium ion, lithium ion, hydrogen ion, chloride ion, magnesium ion, ammonium ion, hydrogen peroxide, ascorbic acid, glucose, cholesterol, uric acid, esterified cholesterol, urea, BUN (blood urea nitrogen), creatinine, creatine, triglycerides, lactate, lactate dehydrogenase, creatine kinase, alkaline phosphatase, creatine kinase-MB, alanine transaminase, aspartate transaminase, bilirubin, amylase, lipase, vitamin K or other clotting factors, anti-clotting factors such as warfarin and heparin, troponin, CrCl microalbuminuria, hs-CRP, CD40L, BNP, NT-proBNP (as described in Morrow & Braunwald, “Future of Biomarkers in Acute Coronary Syndromes: Moving Toward a Multimarker Strategy,” Circulation 108:250-52 (2003)), carcinoembryonic antigen or other tumor antigens, illegal drugs, and various reproductive hormones such as those associated with ovulation or pregnancy. Examples of methods for fabricating biosensor are described for example in U.S. Pat. No. 5,200,051 to Cozzette, et al. and U.S. Patent Application Publications No. 2006/0076236 to Shah et al., and No. 2006/0025748 to Ye et al., which are incorporated herein by reference.

In one embodiment, the reservoir contents comprise at least one sensor indicative of a physiological condition in the patient. For example, the sensor could monitor the concentration of glucose, urea, lactate, calcium, or a hormone present in the blood, plasma, interstitial fluid, vitreous humor, or other bodily fluid of the patient. See, e.g. U.S. Patent Application Publication No. 2005/0096587 to Santini et al., which is hereby incorporated by reference. Information from the sensor could be used, for example, to actively control insulin release from the same device or from a separate insulin delivery device (e.g., a conventional insulin pump, either an externally worn version or an implanted version). Other embodiments could sense other analytes and delivery other types of drugs in a similar fashion.

In one embodiment, the device is used in an ex vivo application to sense critical analytes or compounds. For example, sensors can be included in a dialysis cassette to monitor critical analytes or compounds during dialysis. In one case, the reservoir devices are integrated into a dialysis cassette and contain sensors. See, for example, U.S. Pat. No. 6,887,214 to Levin, which is incorporated herein by reference, which describes monitoring critical analytes or compounds such as metabolites, toxic materials, anti-coagulants, drugs, renal function indicators, phosphate, or biomarkers. A signal from the sensor may be transmitted (by any number of means, including hardwired or telemetry) to a separate molecule delivery device, which could also be located in a dialysis cassette.

In another embodiment, the sensor may be adapted for the detection of airborne analytes. Such embodiments could be useful, for example, in military and homeland defense applications.

In yet another embodiment, the secondary device may be a MEMS device known in the art, such as a pressure sensor, an accelerometer, a gyroscope, a resonator, or the like.

Several options exist for receiving and analyzing data obtained with secondary devices located within the primary (multi-reservoir) device. The primary devices may be controlled by local microprocessors or remote control. Biosensor information may provide input to the controller to determine the time and type of activation automatically, with human intervention, or a combination thereof. For example, the operation of the device can be controlled by an on-board (i.e., within the package) microprocessor. The output signal from the device, after conditioning by suitable circuitry if needed, will be acquired by the microprocessor. After analysis and processing, the output signal can be stored in a writeable computer memory chip, and/or can be sent (e.g., wirelessly) to a remote location away from the reservoir device. Power can be supplied locally by a battery or remotely by wireless transmission. See, e.g., U.S. Patent Application Publication No. 2002/0072784. In one example, the electrical signal from a biosensor can be measured, e.g., by a microprocessor/controller, which then can transmit the information to a remote controller, another local controller, or both. For example, the system can be used to relay or record information on the patient's vital signs or the implant environment, such as drug concentration. Such information could be relayed to the patient's physician via the internet, telephone, or radio signal.

A device or system may have reservoir contents that include both a drug for release and a sensor/sensing component. For example, the sensor or sensing component can be located in a reservoir or can be attached to the device housing or located in another device. The sensor can operably communicate with the device, e.g., through a microprocessor, to control or modify the drug release variables, including dosage amount and frequency, time of release, effective rate of release, selection of drug or drug combination, and the like. The sensor or sensing component detects (or not) the species or property at the site of ex vivo release and further may relay a signal to the microprocessor used for controlling release from the device. Such a signal could provide feedback on and/or finely control the release of a drug. In another embodiment, the device includes one or more biosensors (which may be sealed in reservoirs until needed for use) that are capable of detecting and/or measuring signals within the body of a patient.

Reservoir Caps and Control/Activation Means

As used herein, the term “reservoir cap” refers to a membrane, thin film, or other structure suitable for separating the contents of a reservoir from the environment outside of the reservoir, but which is intended to be removed, disintegrated, or permeabilized at a selected time to open the reservoir and expose its contents. Selectively removing or disintegrating the reservoir caps causes the contents of the reservoir to be exposed to the environment. As used herein, the term “disintegrate” includes degrading, dissolving, rupturing, fracturing or some other form of mechanical failure, as well as a loss of structural integrity due to a chemical reaction (e.g., electrochemical degradation) or phase change (e.g., melting) in response to a change in temperature, unless a specific one of these mechanisms is indicated. The disintegration of the reservoir cap may be by electrochemical activation as described in U.S. Pat. No. 5,797,898, by thermal activated from a separate heat source as described in U.S. Pat. No. 6,527,762, or by electrothermal ablation as described in U.S. Patent Application Publication No. 2004/0121486. These patent documents are incorporated herein by reference. As used herein, the term “environment” refers to the environment external to the reservoirs, including biological fluids and tissues at a site of implantation, air, carrier fluids, physiological fluids, and particulates present during storage or ex vitro use of a device as in transdermal or dialysis applications.

In a preferred embodiment, a discrete reservoir cap completely covers one of the reservoir's openings. In another embodiment, a discrete reservoir cap covers two or more, but less than all, of the reservoir's openings.

In actively controlled devices, the reservoir cap may include essentially any material that can be disintegrated or permeabilized in response to a suitable, applied stimulus (e.g., electric field or current, magnetic field, change in pH, or by thermal, chemical, electrochemical, or mechanical means). Non-limiting examples of suitable reservoir cap materials include gold, titanium, platinum, tin, silver, copper, zinc, alloys, and eutectic materials such as gold-silicon and gold-tin eutectics.

In one embodiment, the reservoir caps are electrically conductive and non-porous. In a preferred embodiment, the reservoir caps are in the form of a thin metal film. In another embodiment, the reservoir caps are made of multiple metal layers, such as a multi-layer/laminate structure of platinum/titanium/platinum. For example, the top and bottom layers may be selected for adhesion layers on (typically only over a portion of) the reservoir caps to ensure that the caps adhere to/bonds with both the substrate area around the reservoir openings, reservoir cap supports (if provided), and a dielectric overlayer (if provided). In one case, the reservoir cap structure is titanium/platinum/titanium/platinum/titanium, where the top and bottom layers serve as adhesion layers, and the platinum layers provide extra stability/biostability and protection to the main, central titanium layer. The thickness of these layers could be, for example, about 300 nm for the central titanium layer, about 40 nm for each of the platinum layers, and between about 10 and 15 nm for the adhesion titanium layers.

In passive devices, the reservoir caps are formed from a material or mixture of materials that degrade, dissolve, or disintegrate over time, or that do not degrade dissolve, or disintegrate, but are permeable or become permeable to molecules or energy. Representative examples of reservoir cap materials include polymeric materials and various types of semi-permeable membranes, and non-polymeric materials such as porous forms of metals (e.g., trabecular metal, a porous tantalum), semiconductors, and ceramics. Passive semiconductor reservoir cap materials include nanoporous or microporous silicon membranes. The reservoir cap material may be a porous silicon, such as a nanoporous silicon membrane (e.g., NANOGATE™ by Imedd Inc. or a nanostructured porous silicon (e.g., BIOSILICON™ by Psividia Ltd.) NANOGATE™ is used as a non-degradable drug diffusion membrane, whereas BIOSILICON™ is used as a degradable matrix to release drug. The reservoir caps may be non-porous and formed of a bioerodible or biodegradable material, known in the art, such as a synthetic polymer, e.g., a polyester (such as PLGA), a poly(anhydride), or a polycaprolactone.

In one passive embodiment, release is initiated by degradation of the reservoir upon exposure to the carrier fluid. The chemical nature of the fluid, e.g., acid versus basic or polar versus non-polar, may cause the reservoir cap material to degrade or dissolve. Once the cap material is completely dissolved, the molecules will be released into the carrier fluid flowing adjacent to the reservoir opening. The fluid may be a liquid that causes the disintegration of the release system or the cap material or both.

The device may include a controller that facilitates and controls reservoir opening, e.g., for disintegrating or permeabilizing the reservoir caps at selected times. The control means may include the structural components and electronics (e.g., circuitry and power source) for powering and for controlling the time at which release or exposure of the reservoir contents is initiated.

The control means can take a variety of forms. In one embodiment, the reservoir cap may comprise a metal film that is disintegrated by electrothermal ablation as described in U.S. Patent Application Publication No. 2004/0121486 A1, which is incorporated herein by reference, and the control means includes the hardware, electrical components, and software needed to control and deliver electric energy from a power source (e.g., battery, storage capacitor) to the selected reservoir caps for actuation, e.g., reservoir opening. For instance, the device can include a source of electric power for applying an electric current through an electrical input lead, an electrical output lead, and a reservoir cap connected therebetween in an amount effective to disintegrate the reservoir cap. Power can be supplied to the control means of the multi-cap reservoir system locally by a battery, capacitor, (bio)fuel cell, or remotely by wireless transmission, as described for example in U.S. Patent Application Publication No. 2002/0072784. A capacitor can be charged locally by an on-board battery or remotely, for example by an RF signal or ultrasound. The device may use acoustic communication and/or powering means, such as described in U.S. Pat. No. 7,024,248 to Penner et al., which is incorporated herein by reference.

In one embodiment, the control means includes an input source, a microprocessor, a timer, a demultiplexer (or multiplexer). The timer and (de)multiplexer circuitry can be designed and incorporated directly onto the surface of the substrate during fabrication. In another embodiment, some of the components of the control means are provided as a separate component, which can be tethered or untethered to the reservoir portion of the device. For instance, the controller and/or power source may be physically remote from, but operably connected to and/or in communication with, the multi-cap reservoir device. In one embodiment, the operation of the multi-cap reservoir system will be controlled by an on-board (e.g., within an implantable device) microprocessor. In another embodiment, a simple state machine is used, as it may be simpler, smaller, and/or use less power than a microprocessor.

In one embodiment utilizing electrothermal ablation, the reservoir cap is formed of a conductive material adapted to have an electrical current passed through it to electrothermally ablate it. The reservoir cap is operably (i e., electrically) connected to an electrical input lead and to an electrical output lead, to facilitate flow of an electrical current through the reservoir cap. When an effective amount of an electrical current is applied through the leads and reservoir cap, the temperature of the reservoir cap is locally increased due to resistive heating, and the heat generated within the reservoir cap increases the temperature sufficiently to cause the reservoir cap to be electrothermally ablated and ruptured. In this embodiment, the reservoir cap is formed of an electrically conductive material and the control circuitry comprises an electrical input lead connected to the reservoir cap, an electrical output lead connected to the reservoir cap, wherein the reservoir cap is ruptured by application of an electrical current through the reservoir cap via the input lead and output lead. In various embodiments, (i) the reservoir cap and the input and output leads may be designed to provide upon the application of electrical current an increase in electrical current density in the reservoir cap relative to the current density in the input and output leads, (ii) the material forming the reservoir cap has a different electrical resistivity, thermal diffusivity, thermal conductivity, and/or a lower melting temperature than the material forming the input and output leads, or (iii) the reservoir cap and the input and output leads are designed to provide upon the application of electrical current an increase in electrical current density in the reservoir cap relative to the current density in the input and output leads, and the material forming the reservoir cap has a different electrical resistivity, thermal diffusivity, thermal conductivity, and/or a lower melting temperature than the material forming the input and output leads.

Preferably, the control circuitry further comprises a source of electric power for applying the electrical current. Representative examples of suitable reservoir cap materials include gold, copper, aluminum, silver, platinum, titanium, palladium, various alloys (e.g., Au—Si, Au—Ge, Pt—Ir, Ni—Ti, Pt—Si, SS 304, SS 316), and silicon doped with an impurity to modulate the conductivity/resistivity because one can use the impurity to increase or decrease the conductivity or resistivity of the silicon, as known in the art. In one embodiment, the reservoir cap is in the form of a thin metal film. In one embodiment, the reservoir cap is part of a multiple layer structure, for example, the reservoir cap can be made of multiple metal layers, such as a multi-layer/laminate structure of platinum/titanium/platinum.

In another embodiment, the reservoir opening is closed by a reservoir cap comprising a dielectric or ceramic film layer and the actuation means comprises (i) an electrically conductive layer on top of the dielectric or ceramic film layer, and (ii) power source and control circuitry for delivering an electric current through the electrically conductive layer in an amount effective to rupture the dielectric or ceramic film layer, wherein the rupture is due to thermal expansion-induced stress on the dielectric or ceramic film layer. The electrically conductive layer and the actuation means can be designed thermally ablate the electrically conductive layer or the electrically conductive layer could remain, in whole or in part, after rupturing the dielectric or ceramic film layer, depending on the particular design for opening/actuation the release of drug from the reservoir.

In one embodiment, release may be in response to electrochemical stimulation. The application of an electrical potential causes the reservoir cap material to dissolve, providing for the release of the molecules into the liquid carrier fluid flowing adjacent to the reservoir opening. In a preferred embodiment, the electric current would be modulated, rather than maintained at a constant value.

In one embodiment, disintegration of the reservoir cap involves rupturing the reservoirs cap by application of a mechanical force generated from within or applied from outside of the reservoir. In such embodiments, the reservoir cap may be formed of a thin film of a metal or other material. In use, the mechanically rupturable reservoir caps may be ruptured by the pressure created by a pressurized reservoir pump such as an elastic bladder or a syringe pump, for example. The rupturable material can be selected from essentially any suitable brittle or fracturable material, such as titanium, tungsten, silicon, glass, or the like. The rupturable material could also be another type of material, such as a rubber or an elastomeric material with one or more defects engineered into it which would cause the reservoir cap to fail by tearing/rupture.

In one embodiment, the device includes a substrate having a two-dimensional array of reservoirs arranged therein, reservoir contents contained in the reservoirs, discrete anode reservoir caps covering each of the reservoirs, cathodes positioned on the substrate near the anodes, and means for actively controlling disintegration of the reservoir caps. The means includes a power source and circuitry to control and deliver an electrical potential; the energy drives a reaction between selected anodes and cathodes. Upon application of a potential between the electrodes, electrons pass from the anode to the cathode through the external circuit causing the anode material (reservoir cap) to oxidize and dissolve into the surrounding fluids, exposing or releasing the reservoir contents. The microprocessor directs power to specific electrode pairs through a demultiplexer as directed by an EPROM, remote control, or biosensor. Examples of reservoir cap materials in this embodiment include gold, silver, copper, and zinc.

Possible reservoir opening and release control methods are further described in U.S. Pat. No. 5,797,898, No. 6,527,762, and No. 6,491,666, No. 6,808,522, No. 6,730,072, No. 6,773,429, No. 6,123,861; U.S. Patent Application Publication Nos. 2004/0121486, 2002/0107470 A1, 2002/0072784 A1, 2002/0138067 A1, 2002/0151776 A1, 2002/0099359 A1, 2002/0187260 A1, 2003/0010808 A1, 2002/0099359 A1, 2004/0082937 A1, and 2004/016914 A1; PCT WO 2004/022033 A2; and PCT WO 2004/026281, as well as in U.S. Patent Application Publications No. 2006/0105275 A1, No. 2006/0057737 A1, No. 2005/0055014 A1, and No. 2006/0171989 A1, all of which are incorporated by reference herein.

The reservoir control means can provide intermittent or effectively continuous release of the drug formulation. The particular features of the control means depend on the mechanism of reservoir cap activation described herein. For example, the control means can include an input source, a microprocessor, a timer, a demultiplexer (or multiplexer), and a power source. The power source provides energy to activate the selected reservoir, e.g., to trigger release of the drug formulation from the particular reservoir desired for a given dose. For example, the operation of the reservoir opening system can be controlled by an on-board microprocessor. The microprocessor can be programmed to initiate the disintegration or permeabilization of the reservoir cap at a pre-selected time or in response to one or more of signals or measured parameters, including receipt of a signal from another device (for example by remote control or wireless methods) or detection of a particular condition using a sensor such as a biosensor. In another embodiment, a simple state machine is used, as it typically is simpler, smaller, and/or uses less power than a microprocessor. The device can also be activated or powered using wireless means, for example, as described in U.S. 2002/0072784 A1 to Sheppard et al., which is incorporated herein by reference.

In one embodiment, the control means includes a microprocessor, a timer, a demultiplexer (or multiplexer), and an input source (for example, a memory source, a signal receiver, or a biosensor), and a power source. The timer and demultiplexer circuitry can be designed and incorporated directly onto the surface of the substrate during electrode fabrication, or may be incorporated in a separate substrate/device body. The microprocessor translates the output from memory sources, signal receivers, or biosensors into an address for the direction of power through the demultiplexer to a specific reservoir on the device. Selection of a source of input to the microprocessor such as memory sources, signal receivers, or biosensors depends on the microchip device's particular application and whether device operation is preprogrammed, controlled by remote means, or controlled by feedback from its environment (i.e., biofeedback). For example, a microprocessor can be used in conjunction with a source of memory such as erasable programmable read only memory (EPROM), a timer, a demultiplexer, and a power source such as a battery or a biofuel cell. A programmed sequence of events including the time a reservoir is to be opened and the location or address of the reservoir is stored into the EPROM by the user. When the time for exposure or release has been reached as indicated by the timer, the microprocessor sends a signal corresponding to the address (location) of a particular reservoir to the demultiplexer. The demultiplexer routes an input, such as an electric potential or current, to the reservoir addressed by the microprocessor. In another embodiment, the electronics are included on the substrate/chip itself, for example, where the electronics are based on diode or transistor technology known in the art.

In one preferred embodiment, the electronics are separable from the reservoir device, such that they are reusable with the multi-reservoir pump devices. The cost to use a multi-reservoir pump device system like this would be significantly less than a system where the electronics were not separable and only could be used once.

Pump

The substance contained in the reservoirs may be directly or indirectly pumped out of the multi-reservoir pump device using a variety of pump known in the art, depending on the particular application. The pump may be essentially any pumping apparatus that causes a carrier fluid to flow through and out of the multi-reservoir pump device. The pump may be one that provides an in-and-out flow, as with a membrane actuator or a synthetic jet type application, as described in U.S. Pat. No. 6,056,204, which is incorporated herein by reference. The pump may be or include an elastic bladder, a syringe pump, a membrane/diaphragm pump, a piston pump with gas generating means, or a peristaltic pump containing a carrier fluid.

In one embodiment, the pump pumps the carrier fluid across one or more surfaces of the substrate and reservoir caps or reservoir openings. For instance, a carrier fluid may be pumped so that it flows into a flow channel adjacent to a reservoir cap which is opened to release or expose the reservoir contents into the carrier fluid. In another embodiment, the pump provides backpressure on a flexible membrane covering an opening of the reservoir opposite a reservoir cap which may be disintegrated or made permeable to empty the molecules from the reservoirs. In yet another embodiment, the pump provides a carrier fluid through the reservoir which provides both backpressure to empty the chemical substances from the reservoirs and also a diluent in which the molecules may be dissolved.

The pump may be a peristaltic micropump. In one case, the pump may be driven by piezoelectric diaphragm actuators and may include back-pressure independent volumetric dosing with a pressure sensor for monitoring the dosing process and detecting catheter occlusions, as described in Geipel, et al., “Design of an Implantable Active Microport System for Patient Specific Drug Release” Proc. 24^{th} LASTED Int'l Multi-Conference Biomedical Engineering (Feb. 15-17, 2006, Innsbruck, Austria).

In a preferred embodiment, the pump can be provided within a device housing also containing the reservoir device. See, e.g., U.S. Pat. No. 5,709,534 to O'Leary and U.S. Pat. No. 5,056,992 to Simons, which are incorporated herein by reference. In some embodiments, the pump may produce sufficient turbulence to mix the drug molecules from the reservoir and the carrier fluid sufficient to form a solution or ordered mixture. Sufficient turbulence also may be created by incorporating baffles within the flow channel and/or by adding a static or dynamic mixer/agitator.

Carrier Fluid

The carrier fluid can be of essentially of any composition in a fluid form suitable for being pumped in the devices described herein. As used herein, the term “fluid” includes liquids, gases, supercritical fluid, solutions, suspensions, gels, and pastes. In preferred embodiments, the fluid is a non-gas, i.e., primarily includes one or more liquids, depending upon the particular device design and application.

Representative examples of suitable carrier fluids for medical applications include natural biological fluids and other physiologically acceptably fluids such as water, saline solution, sugar solution, blood plasma, and whole blood, as well as oxygen, air, nitrogen, and inhalation propellants. The choice of carrier fluid depends on the particular medical application, for example, transdermal drug delivery or sensing applications, dialysis applications, and the like.

In non-medical applications, the carrier fluid also can be selected from a wide range of fluids. Representative examples of suitable carrier fluids for use in fragrance release systems include water, organic solvents (such as ethanol or isopropyl alcohol), aqueous solutions, and mixtures of any of these. Representative examples of suitable carrier fluids for use in beverage additive systems include beverages or beverage bases of any type, such as water (both carbonated and non-carbonated), sugar solutions, and solutions of artificial sweeteners. In in vitro analytical or diagnostic applications, the carrier fluid may be essentially any chemical fluid. Examples include environmental samples of air or water, industrial or laboratory process sampling analysis, fluid samples to be screened in quality control assessments for food, beverage, and drug discovery, and combinatorial screening fluids.

The carrier fluid may be contained within the pump device or it may be stored in/provided from a separate source. For example, in some embodiments, the pump may include an elastic bladder or a syringe and the carrier fluid may be contained within the elastic bladder or syringe. In one case, the pump may provide backpressure to empty the reservoir contents into a carrier fluid flowing across the reservoir openings from a carrier fluid source.

Other Device or System Components

Device Packaging and Housing

Embodiments of the reservoir device may be packaged with the control electronics and power supply as described in U.S. Pat. No. 6,827,250 to Uhland et al., U.S. Patent Publication No. 2005/0050859 to Coppeta et al., and U.S. Patent Application Publication No. 2006/0115323 by Coppeta et al., which are incorporated herein by reference.

The reservoir device and pump may be contained with a device housing for ease of handling and protection of the components. The device housing may be formed from a variety of materials, such as polymers, metals, ceramics, and combinations thereof. In preferred embodiments, the housing is formed of biocompatible materials, such as stainless steel, inert, or hypoallergenic materials known in the art. In transdermal device embodiments, the skin-contacting surface desirably is flexible and hypoallergenic. The housing may further include other components, such as means for delivering an anesthetic or permeation enhancer. Alternatively, the reservoir device may be remote from the pump/carrier fluid source and connected together with a fluid conduit such as a flexible tube.

Securement Means

For embodiments in which the multi-reservoir pump device is intended for use in transdermal drug delivery or sensing applications, the device preferably is suitably (removably) secured to the site for the intended duration of use. Such securement means can be essentially any device or system known in the art for securing objects to the skin of a patient. For example, the securement means can include one or more biocompatible adhesives, straps, or elastic bands. In one embodiment, the securing means is provided along the periphery of a housing of the device. Adhesive securing means can be, or can be readily adapted from, those known in the art for securing transdermal patches, such as those currently used in commercially available transdermal patches. See, e.g., U.S. Pat. No. 6,632,906, which is incorporated herein by reference.

In one embodiment, the adhesive is provided on a thin permeable material, such as a porous polymer layer, or a woven or non-woven fabric layer, which is adjacent the reservoir caps or the transport means. In one embodiment, the adhesive layer is permeable to the one or more pharmaceutical agents. In one embodiment, the polymer layer comprises a hydrogel. In a preferred embodiment, the securing means comprises a pressure sensitive adhesive, as known in the art.

Needle—Transdermal Delivery Component

In embodiments where the medical device comprises a transdermal multi-reservoir pump device, the device includes one or more conventional hypodermic needles, one or more microneedles, and/or one or more other components for transdermally delivering the combined carrier fluid/drug into/through a patient's skin. The needle may be solid or hollow. The needle may be made of a porous material. It may have a cylindrical or barb- or blade-like shape.

Examples of microneedles suitable for transdermal drug delivery and analyte sensing are described in U.S. Pat. No. 6,743,211, U.S. Pat. No. 6,661,707, U.S. Pat. No. 6,503,231, and U.S. Pat. No. 6,334,856, all to Prausnitz et al., and in U.S. Pat. No. 6,230,051 and U.S. Pat. No. 6,219,574, both to Cormier et al, all of which are incorporated herein by reference. In optional embodiments, the transdermal delivery component may include devices known in the art for driving fluid drug formulations through the stratum corneum by diffusion, capillary action, electroosmosis, electrophoresis, convection, magnetic field, ultrasound, or a combination thereof. These driving devices may be used together with, or in place of, one or more needles or microneedles.

The dimensions of the microneedles are designed for the particular application. The microneedle may be hollow or solid. It may be tapered or straight. The microneedle length typically is selected taking into account both the portion that would be inserted into the biological tissue and the (base) portion that would remain uninserted. The cross-section, or width, is tailored to provide, among other things, the mechanical strength to remain intact for the delivery of the drug while being inserted into the tissue, while remaining in place during drug delivery, and while being removed (unless designed to break off, dissolve, or otherwise not be removed). The microneedle may have a length of about 50 μm to about 2000 μm. In one embodiment, the microneedle may have a length of about 150 μm to about 2000 μm, about 300 μm to about 2000 μm, about 300 μm to about 1500 μm, about 300 μm to about 1000 μm, or about 300 to about 750 μm. In one embodiment, the length of the microneedle is about 500 μm. The base portion of the microneedle, at its widest part, may have a width or cross-sectional dimension of about 20 μm to about 500 μm, about 50 μm to about 400 μm, or about 100 μm to about 250 μm. For a hollow microneedle, the maximum outer diameter or width may be about 50 μm to about 400 μm, with an aperture diameter of about 5 μm to about 100 μm. The microneedle may be fabricated to have an aspect ratio (width:length) of about 1:1.5 to about 1:10. Other lengths, widths, and aspect ratios are envisioned.

The microneedle may be formed/constructed of different biocompatible materials, including metals, glasses, semi-conductor materials, ceramics, or polymers. Examples of suitable metals include pharmaceutical grade stainless steel, gold, titanium, nickel, iron, gold, tin, chromium, copper, and alloys thereof The microneedle may be formed of a coated or uncoated metal, silicon, glass, or ceramic. The microneedle may include or be formed of a biodegradable or non-biodegradable polymer.

Methods for Manufacture or Assembly

The multi-reservoir devices may be made, for example, using techniques known in the art, particularly the methods described in U.S. Pat. No. 6,123,861 to Santini et al., U.S. Pat. No. 6,808,522 to Richards et al., U.S. Patent Application Publication No. 2004/0121486 to Uhland et al., U.S. Patent Application Publication No. 2006/0057737 to Santini Jr. et al., U.S. Patent Application Publication No. 2006/0105275 to Maloney et al., which are each incorporated herein by reference.

The fabrication methods may use microfabrication and microelectronic processing techniques; however, it is understood that fabrication of device reservoir structures is not limited to materials such as semiconductors or processes typically used in microelectronics manufacturing. For example, other materials, such as metals, ceramics, and polymers, can be used to make the devices. Similarly, other fabrication processes, such as plating, casting, or molding, can also be used to make them.

In one embodiment, reservoirs may be formed using a silicon-on-insulator (SOI) techniques, such as described in S. Renard, “Industrial MEMS on SOI,” J. Micromech. Microeng. 10:245-249 (2000). SOI methods can be usefully adapted to form reservoirs having complex reservoir shapes. SOI wafers behave essentially as two substrate portions that have been bonded on an atomic or molecular-scale before any reservoirs have been etched into either portion. SOI substrates easily allow the reservoirs (or reservoir sections) on either side of the insulator layer to be etched independently, enabling the reservoirs on either side of the insulator layer to have different shapes. The reservoir (portions) on either side of the insulator layer then can be connected to form a single reservoir having a complex geometry by removing the insulator layer between the two reservoirs using methods such as reactive ion etching, laser, ultrasound, or wet chemical etching.

In a preferred embodiment, the device includes at least two substrates portions bonded together as described in U.S. Patent Application Publication No. 2006/0115323 to Coppeta et al. The substrates include at least one cavity (i.e., a reservoir), which may be defined in one or both substrate portions. The space in the sealed cavity may be evacuated or may contain an inert gas or gas mixture (e.g., nitrogen, helium). In one case, the device includes contains a MEMS device, which may be on a third substrate. In another case, at least one of the bonded substrates is formed of a glass and the cavity contains an optical sensor or chemical compound that can be optically interrogated.

Modifications and variations of the methods and devices described herein will be obvious to those skilled in the art from the foregoing detailed description. Such modifications and variations are intended to come within the scope of the appended claims.

We claim:

1. A pump patch device for drug delivery comprising:
a substrate which includes a plurality of discrete reservoirs, each reservoir having at least one reservoir opening;
a drug disposed in the reservoirs;
a pump for delivering a carrier fluid through or adjacent to the at least one opening of each of the reservoirs;
a flow channel for receiving and combining the carrier fluid from the pump and the drug from at least one of the reservoirs to form a fluidized drug; and
at least one needle for delivering the fluidized drug into the skin or another biological tissue of a patient.

2. The device of claim 1, further comprising a first plurality of discrete reservoir caps, each cap closing the at least one reservoir opening of each reservoir.

3. The device of claim 2, further comprising a controller and a power source for actively disintegrating the first plurality of reservoir caps to initiate mixing of the drug with the carrier fluid.

4. The device of claim 3, wherein the controller and the power source are part of a reusable module which can be releasably secured to a drug reservoir array module comprising the substrate, the drug the pump, the flow channel, the at least one needle, and a source of carrier fluid.

5. The device of claim 1, which comprises an array of microneedles.

6. The device of claim 1, wherein the pump comprises a pressurized reservoir or gas generation mechanism.

7. The device of claim 1, wherein the pump comprises a syringe pump or a peristaltic pump.

8. The device of claim 2, wherein each reservoir comprises a second reservoir opening and the second reservoir openings are closed by a second plurality of reservoir caps.

9. The device of claim 8, further comprising a second flow channel wherein carrier fluid can flow through a reservoir following disintegration of the reservoir caps closing the first and second reservoir openings of the reservoir.

10. The device of claim 2, wherein the pump comprises a carrier fluid reservoir which can be pressurized to drive carrier fluid through the flow channel.

11. The device of claim 10, further comprising a pressure manifold with a flexible membrane which, following disintegration of the reservoir cap closing the at least one reservoir opening, pushes against the drug from the side of the reservoir opposed to the reservoir opening in order to displace the drug from the reservoir.

12. The device of claim 1, wherein the drug in the reservoirs is in a solid or gel formulation.

13. The device of claim 1, further comprising a housing for the substrate, the drug the pump, the flow channel, the at least one needle, and a source of carrier fluid, wherein the device further includes means for securing the device to the skin or other biological tissue surface.

14. A method for delivering a drug into the skin or another biological tissue of a patient, the method comprising:
providing a pump patch device that comprises (i) a substrate which includes a plurality of discrete reservoirs, each reservoir having at least one reservoir opening; (ii) a drug disposed in the reservoirs; (iii) a pump comprising a carrier fluid supply, (iv) a flow channel, and (v) at least one needle;
inserting the needle into the patient's skin or other biological tissue;
pumping the carrier fluid from the pump through or adjacent to the at least one opening of each of the reservoirs;
combining in the flow channel the carrier fluid from the pump with the drug from at least one of the reservoirs to form a fluidized drug; and
pumping the fluidized drug through the needle and into the patient.

15. The method of claim 14, wherein the pump patch comprises a plurality of needles and the needles are microneedles.

16. The method of claim 14, wherein the pump patch further comprises a plurality of discrete reservoir caps, each cap closing the at least one reservoir opening of each reservoir.

17. The method of claim 16, wherein the pump patch further comprises a controller and a power source for actively disintegrating the plurality of reservoir caps to initiate the combining of the drug with the carrier fluid in the flow channel.

18. A device for use in dialysis comprising:
a non-disposable module which comprises a pump or pressure generator;
a disposable cassette operably connected to the pump or pressure generator, wherein the cassette includes
a plurality of discrete reservoirs, each having at least one reservoir opening,
reservoir contents located in the reservoirs, which reservoir contents comprise a drug, a sensor or sensor component, or a combination thereof, and
a plurality of discrete reservoir caps, each cap closing the at least one reservoir opening of each reservoir; and
power and control electronics for actively and selectively disintegrating the reservoir caps to expose the reservoir contents to a physiological fluid, a dialysate, or a combination thereof.

19. The device of claim 18, wherein the reservoir contents comprises a sensor or sensor component which can measure or monitor temperature, pH, salt concentration, metabolites, waste products, and/or blood gases of the blood or peritoneal fluid of a dialysis patient while the patient is be dialyzed.

20. The device of claim 18, wherein the reservoir contents comprises a sensor or sensor component which can measure or monitor blood coagulation by measuring the level of one or more anti-coagulants, blood viscosity, clotting time, or a combination thereof.

21. The device of claim 18, wherein the reservoir contents comprises an anti-coagulant or other drug for release.

22. A fluidics connection device comprising:
a first substrate portion which comprises a seating surface, an opposing surface, and at least one microfluidic via therethrough;
a nipple connector which comprises sealing surface and at least one fluid aperture therethrough; and
a compression cold weld seal which attaches the sealing surface of the first substrate portion to the sealing surface of the nipple connector, such that the microfluidic via is aligned in fluid communication with the fluid aperture.

23. The fluidics connection device of claim 22, having a plurality of microfluidic vias and a plurality of corresponding fluid apertures, wherein the interface of each via with its corresponding fluid aperture is surrounded by a separate compression cold weld seal.

24. The fluidics connection device of claim 22, wherein the compression cold weld seal comprises at least one ridge feature on one of the sealing surfaces and at least one groove in the other of the sealing surfaces.

25. The fluidics connection device of claim 22, further comprising a second substrate portion attached by at least one compression cold weld seal to the opposing surface of the first substrate portion, wherein the second substrate comprises a second microfluidic via and/or microfluidic channel.
